(19) United States
(12) Patent Application Publication
(10) Pub. No.: US 2006/0135571 A1
(43) Pub. Date: Jun. 22, 2006

(54) Imidazo(2,1-b)-1,3,4-thiadiazole sulfoxides and sulfones

(76) Inventors: James B. Jaquith, Pincourt (CA); John W. Gillard, Baie D'Urfe (CA); Patrick Bureau, Kirkland (CA); Scott Jarvis, Longueuil (CA)

Correspondence Address: AEGERA THERAPEUTICS INC., 810 CHEMIN DU GOLF, VERDUN, QC, H3E-1A8, CA

(21) Appl. No.: 10/559,234

(22) PCT Filed: Jun. 14, 2004
(86) PCT No.: PCT/CA04/00871

§ 371 Date: Dec. 6, 2005

Related U.S. Application Data
(60) Provisional application No. 60/477,954, filed on Jun. 13, 2003.

(51) Int. Cl. A61K 31/433; C07D 498/02

(52) U.S. Cl. 514/362; 548/126

(57) ABSTRACT

This invention relates to novel imidazo[2,1-b]-1,3,4-thiadiazole sulfoxide and sulfone compounds of Formula (I) and the use of compounds of Formula (I) in the treatment of neuronal disorders of the central and peripheral nervous systems and for the treatment of proliferative diseases such as cancer.

### FIELD OF THE INVENTION

This invention relates to compounds and the use of compounds in the treatment of neuronal disorders of the central and peripheral nervous systems and for the treatment of proliferative diseases such as cancer and inflammation.

### BACKGROUND OF THE INVENTION

The Applicant has previously demonstrated that compounds represented by formula A protect SCG neurons from several neurotoxic insults, including NGF withdrawal and treatment with chemotherapeutics such as Taxol™, cisplatin, and vincristine (PCT Application No. CA02/01942 (WO 03/051890)). Additionally, compounds represented by formula A protect cortical motor neurons from malonate induced death (PCT Application No. CA02/01942 (WO 03/051890)).

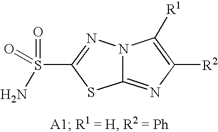

When such agents are administered to mice treated with Taxol™, either during or after a two week dosing period, marked improvements are observed in the animal's general health, weight gain, gait, and nerve conductance as compared to animals treated with Taxol™ alone. Additionally, these compounds aid in the regeneration of neurons damaged as a result of sciatic nerve crush and protect retinal ganglion neurons from ocular stroke (PCT Application No. CA02/01942 (WO 03/051890)).

### SUMMARY OF THE INVENTION

The present invention relates to imidazo[2,1-b]-1,3,4-thiadiazole sulfoxides and sulfones, represented by Formula I:

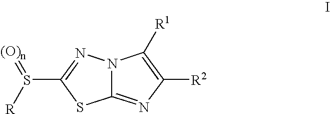

The applicant herein disclose that select compounds represented by Formula I protect SCG neurons from neurotoxic insults such as treatment with anti-NGF antibody, Taxol, and cisplatin. Select compounds of this class have also been shown to kill various cancer cell lines. These compounds are useful as therapeutic agents for the treatment of neurodegenerative disease and/or for the treatment of proliferative diseases such as cancer.

This invention provides compounds of Formula I and the use of compounds of Formula I for the prevention of neuronal cell loss or the treatment of nerve cell or axonal degradation, in either the central or peripheral nervous systems (CNS and PNS, respectively), resulting from such diseases as Alzheimer's, Huntington's, Parkinson's, muscular dystrophy, diabetes, HIV, from ischemic insults such as stroke in the brain (CNS), retinal ganglion loss following acute ocular stroke or hypertension as in glaucoma, and from infection by viruses such as Hepatitis C and Herpes Simplex, or for the treatment of neuropathies resulting from the use of chemotherapeutic agents used in the treatment of HIV and proliferative disease such as cancer, for the treatment of inflammatory diseases. Additionally, compounds of Formula I are useful in the treatment of proliferative diseases such as cancer and inflammation where uncontrolled cellular proliferation results in tumor formation and/or Ussue damage as a result of inflammation.

The imidazo[2,1-b]-1,3,4-thiadiazole sulfoxides and sulfones of the present invention are represented by Formula I: 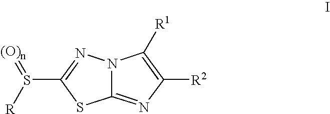
or a pharmaceutically acceptable salts thereof wherein:

n is 1 or 2;

R is selected from the group consisting of
i. substituted or unsubstituted C (1-8) alkyl, fluoroalkyl, substituted and unsubstituted aralkyl, substituted and unsubstituted aryl, substituted and unsubstituted heteroaryl; and
ii. 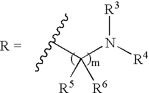
wherein
   m is an integer between 1 and 5; and
   R³ and R⁴ are independently selected from the groups consisting of hydrogen, substituted or unsubstituted C (1-8) alkyl, fluoroalkyl, substituted and unsubstituted aralkyl, substituted and unsubstituted aryl, substituted and unsubstituted heteroaryl, substituted or unsubstituted C (1-8) alkylcarbonyl, substituted or unsubstituted C (1-8) alkylaminocarbonyl, substituted or unsubstituted arylaminocarbonyl, substituted or unsubstituted heteroarylaminocarbonyl, substituted or unsubstituted C (1-8) alkylsulfonyl, substituted or unsubstituted arylsulfonyl, substituted or unsubstituted heteroarylsulfonyl, or R³ and R⁴ are joined to form a substituted or unsubstituted cycloalkyl or cycloheteroalkyl ring system;
   R⁵ and R⁶ are independently chosen from hydrogen, substituted or unsubstituted C (1-8) alkyl, or R⁵ and R⁶ are joined to form a cydoalkyl or cycloheteroalkyl ring system; and

R¹ and R² are independently selected from the group consisting of

(i) fluoro, C(1-6)-alkyl, substituted and unsubstituted C(6-16)-aryl, substituted and unsubstituted heteroaryl, substituted and unsubstituted biphenyl, substituted and unsubstituted diphenyl ether, substituted and unsubstituted coumarinyl, and adamantyl;

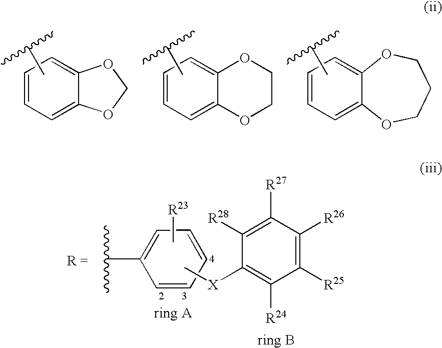

wherein

X is represented by a bond, O or S(O)ₙ, wherein n=0, 1, or 2, and is attached to ring A at the 2, 3, or 4 position;

R²³ on ring A are selected from the group consisting of H, halogen, C(1-8)alkyl, C(1-8) alkoxy and represents up to 4 substitutions;

R²⁴ through R²⁸ of ring B is independently selected from the group consisting of H, halogen, C(1-8) alkyl, C(1-8) flouroalkyl, C(1-8) alkoxy,

wherein any two adjacent R groups may be combined to form members of a fused aryl, substituted aryl, heteroaryl, or substituted heteroaryl, ring system; and 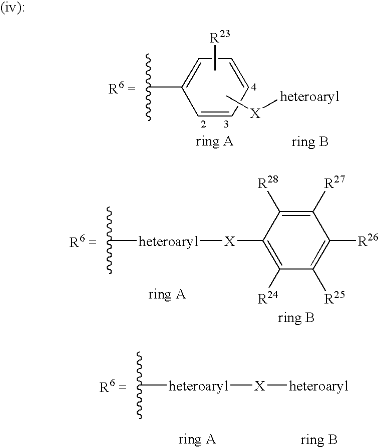
wherein

X is represented by a bond, O or S(O)ₙ, wherein n=0, 1, or 2;

R²³ on ring A is selected from the group consisting of H, halogen, C91-8) alkyl, C(1-8) alkoxy and represents up to 4 substitutions;

the heteroaryl ring systems of ring A and B contain at least on heteroatom and are substituted or unsubstituted;

R²⁴ through R²⁸ of ring B are independently selected from the group consisting of H, halogen, C(1-8) alkyl, C(1-8) flouroalkyl, C(1-8) alkoxy; and

wherein any two adjacent R groups may be combined to form members of a fused aryl, substituted aryl, heteroaryl, or substituted heteroaryl, ring system.

### BRIEF DESCRIPTION OF FIGURE

FIG. 1 illustrates in vitro killing of human neuroblastomas SK-N-AS and SH-SY5Y and Daoy medulloblastoma cells with compound 28. Human neuroblastomas SK-N-AS and SH-SY5Y and Daoy medulloblastoma cells were cultured in the presence of compound 28 and cellular viability was measured using sulforhodamine B. Compound 28 potently kills these cell lines in vitro at concentrations as low as 5 μM.

### DETAILED DESCRIPTION OF THE INVENTION

The invention provides imidazo[2,1-b]-1,3,4-thiadiazole sulfoxides and sulfones, and derivatives thereof, as well as methods of treatment, and uses of such compounds. Further, methods of preparation of such compounds are provided.

In the definitions of the groups of Formula I, C(1-8) alkyl means a straight-chain or branched alkyl group having 1 to 8 carbon atoms, such as methyl, ethyl, propyl, iso-propyl, n-butyl, iso-butyl, sec-butyl, tert-butyl, pentyl, iso-amyl, neopentyl, 1-ethylpropyl, hexyl, and octyl. The C(1-8) alkyl moiety of C(1-8) alkoxy, C(1-8) alkylsulfonyl, C(1-8) alkoxylcarbonyl, C(1-8) alkylaminocarbonyl has the same meaning as lower alkyl defined above. The acyl moiety of the acyl and the acyloxy group means a straight-chain or branched alkanoyl group having 1 to 18 carbon atoms, such as acetyl, propanoyl, butyryl, valeryl, pivaloyl and hexanoyl, and arylcarbonyl group described below, or a heteroarylcarbonyl group described below. The aryl moiety of the aryl, the arylcarbonyl and arylaminocarbonyl groups means a group having 6 to 16 carbon atoms such as, but not limited to. phenyl, biphenyl, naphthyl, or pyrenyl. The heteroaryl moiety of the heteroaryl and the heteroarylcarbonyl groups contain at least one hetero atom from O, N, and S, such as, but not limited to pyridyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl. The aralkyl moiety of the aralkyl and the aralkyloxy groups having 7 to 15 carbon atoms, such as, but not limited to, benzyl, phenethyl, benzhydryl, and naphthylmethyl. The heteroaralkyl moiety of the heteroaralkyl and the heteroaralkyloxy groups having 7 to 15 carbon such as, but not limited to, pyridylmethyl, quinolinylmethyl, and iso-quinolinylmethyl. The substituted C(1-8) alkyl group has 1 to 3 independently-substitutuents, such as but not limited to hydroxyl, C(1-8) alkyloxy, carboxyl, C(1-8) alkylcarbonyl, nitro, amino, mono- or di-C(1-8) alkylamino, dioxolane, dioxane, dithiolane, and dithione. The C(1-8) alkyl moiety of the substituted C(1-8) alkyl, and the C(1-8) alkyl moeity of the C(1-8) alkoxy, the C(1-8) alkoxycarbonyl, and the mono- and di-lower alkylamino in the substituents of the substituted C(1-8) alkyl group have the same meaning as C(1-8) alkyl defined above. The substituted aryl, the substituted heteroaryl, the substituted aralkyl, and the substituted heteroaralkyl groups each has 1 to 5 independently-selected substituents, such as but not limited to C(1-8) alkyl, hydroxy, C(1-8) alkoxy, carboxy, C(1-8) alkoxycarbonyl, nitro, amino, mono or di-C(1-8) alkylamino, azido, and halogen. The C(1-8) alkyl moiety of the C(1-8) alkyl, the C(1-8) alkoxy, the C(1-8) alkylamino, and the mono- and di-C(1-8) alkylamino groups amoung the susbtituents has the same meaning as C(1-8) alkyl defined above. The heterocyclic group formed with a nitrogen atom includes rings such as, but not limited to, pyrrolyl, piperidinyl, piperidino, morpholinyl, morpholino, thiomorpholino, N-methylpiperazinyl, indolyl, and isoindolyl. The cycloalkyl moeity means a cycloalkyl group of the indicated number of carbon atoms, containing one or more rings anywhere in the structure, such as cycloalkyl groups include cyclopropyl, cyclopropylmethyl, cyclobutyl, cyclopentyl, cyclohexyl, 2-norbornyl, 1-adamantyl and the like. The fluoroalkyl moiety means a lower fluoroalkyl group in which one or more hydrogens of the corresponding C(1-8) alkyl group, as defined above, is replaced by a fluorine atom, such as but not limited to CH₂F, CHF₂, CF₃, CH₂CF₃, and CH₂CH₂CF₃.

The substituents are preferably selected from the group consisting of:
1) H, halogen, nitro, cyano, C(1-8) alkyl, C(1-8) fluoroalkyl, aralkyl, aryl, heteroaryl, C(1-8) alkylcarbonyl, arylcarbonyl, heteroarylcarbonyl, azide, B(OH)₂, and adamantyl;
2) XR¹⁹ wherein X═O or S and R¹⁹ is defined as a C(1-8) alkyl, hydroxyl, C(1-4) alkoxy, fluoroalkyl, aryl, heteroaryl, lower alkylcarbonyl, arylcarbonyl, heteroarylcarbonyl, lower alkylaminocarbonyl, and arylaminocarbonyl; and
3) NR¹⁴R¹⁵ wherein R¹⁴ and R¹⁵ are independently defined as C(1-8) alkyl, or wherein R¹⁴ and R¹⁵ are joined to form an alkyl or heteroalkyl ring system
   wherein said C(1-8) alkyl, C(1-8) fluoroalkyl, aralkyl, aryl, heteroaryl, C(1-8) alkylcarbonyl, arylcarbonyl, heteroarylcarbonyl, and C(1-4) alkoxy may be further substituted, preferably by the substituents 1-3 listed above.

Some of the compounds described herein contain one or more chiral centres and may thus give rise to diastereomers and optical isomers. The present invention is meant to comprehend such possible diastereomers as well as their racemic, resolved and enantiomerically pure forms, and pharmaceutically acceptable salts thereof.

The term “subject” or “patient” as used herein may refer to mammals including humans, primates, horses, cows, pigs, sheep, goats, dogs, cats and rodents.

The pharmaceutical compositions of the invention are administered to subjects in effective amounts. An effective amount means that amount necessary to delay the onset of, inhibit the progression of, or halt altogether the onset or progression of, or diagnose the particular condition or symptoms of, the particular condition being treated.

An effective amount for treating a neurological disorder is that amount necessary to affect any symptom or indicator of the condition, and/or reverse, halt or stabilize neuronal degradation and/or cell loss that is responsible for the particular condition being treated. In general, an effective amount for treating neuropathies and neuropathic pain will be that amount necessary to favorably affect the neuropathies and/or neuropathic pain. For example, an effective amount for treating neurodegenerative disease of the CNS, such as Alzheimer's disease is an effective amount to prevent memory loss, but is not limited to the amelioration of any one symptom. Similarly, an effective amount for treating Parkinson's disease or ALS is an amount necessary to favorably effect loss of muscular function and/or control, but is not limited to the amelioration of any one symptom. An effective amount for treating glaucoma and macular degeneration is an effective amount to prevent loss of vision. An effective amount for treating a peripheral neuropathy is an effective amount for preventing the development or halting the progression of PNS sensory or motor nerve dysfunction, but is not limited to these symptoms or effects.

In general, an effective amount for treating a mammalian cancer cell proliferation is that amount necessary to affect any symptom or indicator of the condition, and/or reverse, halt or stabilize mammalian cancer cell proliferation and/or migration that is responsible for the particular condition being treated, with that amount being the amount necessary to favorably affect mammalian cancer cell proliferation in vivo.

When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

A variety of administration routes are available. The particular mode selected will depend, of course, upon the particular condition being treated, the particular drug selected, the severity of the condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal, intradermal or parenteral routes. The term “parenteral” includes subcutaneous, intravenous (IV), intramuscular, or infusion.

Dosage may be adjusted appropriately to achieve desired drug levels, locally or systemically. Generally, daily oral doses of active compounds will be from about 0.01 mg/kg per day to 1000 mg/kg per day. It is expected that IV doses in the range of about 1 to 1000 mg/m² per day will be effective. In the event that the response in a subject is insufficient at such doses, even higher doses (or effective higher doses by a different, more localized delivery route) may be employed to the extent that patient tolerance permits.

The compositions may conveniently be presented in unit dosage form and may be prepared by any of the methods well known in the art of pharmacy. All methods include the step of bringing the conjugates of the invention into association with a carrier that constitutes one or more accessory ingredients. In general, the compositions are prepared by uniformly and intimately bringing the compounds into association with a liquid carrier, a finely divided solid carrier, or both, and then, if necessary, shaping the product.

Compound may be administered as an aqueous and/or non-aqueous solution, being dissolved or suspended in a pharmaceutically acceptable aqueous and/or non-aqueous formulation, prepared by any of the methods well known in the art of pharmacy. These aqueous and/or non-aqueous solutions may contain buffering agents, co-solvents, stabilizers, surfactants, co-solvents and/or encapsulating agents. Buffers and stabilizers are described below, and co-solvents may include HPCD or other encapsulating co-solvents known in the art, PEG and the like.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating solid tumors by placing the implant near or directly within the tumor, thereby affecting localized, high-doses of the compounds of the invention.

When administered, the Formulations of the invention are applied in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such salts include, but are not limited to, those prepared from the following acids: hydrochloric, hydrobromic, sulphuric, nitric, phosphoric, maleic, acetic, salicylic, p-toluenesulfonic, tartaric, citric, methane sulfonic, formic, malonic, succinic, naphthalene-2-sulfonic, benzene sulfonic, and the like. Also, pharmaceutically acceptable salts can be prepared as alkaline metal or alkaline earth salts, such as sodium, potassium or calcium salts, or from organic bases known in the art such as, but not limited to dimethylaminoethanol, ethanolamine, arginine and lysine.

Suitable buffering agents include: phosphate buffers, acetic acid and a salt (1-2% W/V); citric acid and a salt (1-3% W/V); and phosphoric acid and a salt (0.8-2% W/V), as well as others known in the art.

Suitable preservatives include benzalkonium chloride (0.003-0.03% W/V); chlorobutanol (0.3-0.9% W/V); parabens (0.01-0.25% W/V) and thimerosal (0.004-0.02% W/V), as well as others known in the art.

Suitable carriers are pharmaceutically acceptable carriers. The term pharmaceutically acceptable carrier means one or more compatible solid or liquid filler, dilutants or encapsulating substances that are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy. Carrier Formulations suitable for oral, subcutaneous, intravenous, and intramuscular administration etc., are those which are known in the art.

The compounds of the invention may be delivered with other therapeutic agents. The invention additionally includes co-administration of compound I of the invention with other compounds known to be useful in treating neurodegenerative or proliferative diseases. In neurodegenerative disease this is typified by but not limited to, COX-2 inhibitors, NSAIDS, acetylcholinesterase inhibitors for treating AD, such as tacrine, doneprizil, and rivastigmin, and L-dopa for treating PD, and ACE inhibitors and insulin for the treatment of diabetes. In proliferative diseases such as cancer, this is typified by chemotherapeutics such as Taxol, cisplatin, and the vinca alkaloids

In the case of peripheral neuropathy induced by a toxic agent, compound I would be delivered separately before, simultaneously with (ie. independently or in the form of anti-cancer coctails), or after exposure to the toxic agent. Preferably, compound I and the chemotherapeutic agent are each administered at effective time intervals, during an overlapping period of treatment in order to prevent or restore at least a portion of the neurological function destroyed by the neurotoxic or chemotherapeutic agent. The chemotherapeutic can be any chemotherapeutic agent that causes neurotoxicity, such as dideoxyinosine, deoxy cytizine, D4T, cisplatin, etoposide, vincristine, epithilone or its derivatives, or Taxol™/Taxoter™ and derivatives thereof, which are representative of the classes of agents which induce neuropathies.

By “toxic agent” or “neurotoxic agent” is meant a substance that through its chemical action injures, impairs, or inhibits the activity of a component of the nervous system. Such neurotoxic agents include, but are not limited to, neoplastic agents such as vincristine, vinblastine, cisplatin, Taxol™, D4T or other anti-virals, or dideoxy-compounds, eg., dideoxyinosine; alcohol; metals; industrial toxins involved in occupational or environmental exposure; contaminants in food or medicinals; or over-doses of vitamines or therapeutic drugs, eg. Antibiotics such as penicillin or chloramphenicol, or mega-doses of vitamins A, D, or B6.

In the treatment of cancer where compounds represented by formula I are to be used as pro-apoptotic agents for the killing of cancer cells in vivo compound I would be delivered alone, separately before, simultaneously with (ie. independently or in the form of anti-cancer coctails), or after treatment with traditional chemotherapeutics such as, but not limited to, Taxol, Taxoter, cisplatin, the vinca alkaloids, epithilone and derivatives thereof, and 5-fluorouracil.

Neuroprotective and Anti-Cancer Activity of Compounds Represented by Formula I

Compounds represented by Formula I protect SCG neurons from neurotoxic insults such as treatment with anti-NGF antibody, Taxol, and cisplatin. Select compounds of this class have also been shown to kill various cancer cell lines. These compounds are useful as therapeutic agents for the treatment of neurodegenerative disease and/or for the treatment of proliferative diseases such as cancer.

Neurotrophins are critical to the growth, development, and survival of small fiber neurons of the PNS. Superior Cervical Ganglion (SCG) neurons are neurons of the PNS that undergo apoptosis upon Neuronal Growth Factor (NGF) withdrawal. In a typical experiment SCG neurons are cultured in the presence of NGF, which induces survival and neurite out-growth. After 5 days the NGF is removed by either the addition of anti-NGF polyclonal antibody (Sigma) or by repeated washings (4 times) with NGF free media, resulting in the apoptosis of up to 90% of the neurons after 48 hours, as measured by MTS staining. The addition of selected compounds of Formula I to the final cellular media provides upwards of 100% protection, at drug concentrations ranging from 1 to 50 μM (see Example 73).

Taxol™ is regularly used in breast cancer chemotherapy. In cancer cells Taxol™ binds to the cyto-skeletal protein tubulin, thereby inhibiting normal microtubular assembly and inducing cellular apoptosis. Despite its potency as an anti-tumour agent, Taxol™ is also toxic to neurons, inducing dose limiting peripheral neuropathies. The addition of Taxol™ (100 ng/mL) to cultured SCG neurons induces the degradation or loss of upwards of 80% of the neurons. The addition of selected compounds of Formula I to the cellular media, concurrently with Taxol™, protects upwards of 100% of the neurons, at drug concentrations ranging from 1 to 50 μM (see Example 74).

The mechanism of Cisplatin's anti-cancer action is not fully understood, but is believed to involve DNA binding and cleavage. Cisplatin is highly toxic to neurons. The addition of cisplatin (3 μg/mL) to cultured SCG neurons induces apoptosis of upwards of 80% of the neurons. The addition of selected compounds of Formula I to the cellular media, concurrently with cisplatin, protects upwards of 100% of the neurons, at drug concentrations ranging from 1 to 50 μM (see Example 75).

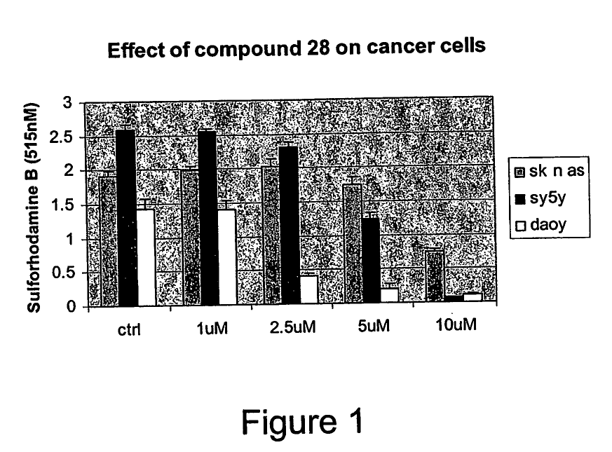

Compound represented by Formula I were tested for anti-cancer properties using the sulforhodamine B assay. Human neuroblastomas SK-N-AS, and SH-SY5Y were plated in 96 well plates at a density of 15 000 cells/well. Daoy medulloblastoma cells were plated at a density of 2000 cells/well. Cells were cultured in RPMI supplemented with 5% fetal bovine serum and test compounds. After 72 hours of culture cell growth was assessed by sulforhodamine B. Cellular protein was precipitated and fixed with trichloroacetic acid and detected by incubation with sulforhodamine B. Bound dye solubilized in trizma base and was assessed by absorbance at 515 nM. Compound 28 caused either cell death or inhibited cellular proliferation of the SK-N-AS and SH-SY5Y neuroblastoma and daoy medulloblastoma cells with ED₅₀s of 7, 5, and 2□M, respectively. These data suggest that selected compound represented by formula I have anti-cancer properties that may be useful in the treatment of human cancers (see Example 76 and FIG. 1).

Select compounds bearing a basic moiety display good aqueous solubility as the corresponding acid salt. For example, compound 56.2HCl is soluble at >100 mg/mL in saline, allowing for the ready application of these compounds via multiple routes of administration either oral or percutaneous.

For any of the compounds having the structure of Formula I which bear similarity to those known in the art, the use of these compounds for treatment and/or prevention of neurological disorders, cancer, inflammation, or symptoms related thereto are encompassed by the invention.

Synthetic Procedures

Compounds of the present invention may be prepared in the following manner. Three General Procedure have been employed.

General Procedure A:

The desired sulfoxides and sulfones were prepared by ‘sulfonamide’ or bromide displacement with a thiol under basic conditions. Intermediates A or B may be conversted to the sulfide intermediate C, by treatment with a primary thiol under basic conditions. Subsequent oxidation of C with an appropriate oxidizing agent provides a mixture of the desired sulfoxide (n=1) and/or sulfone (n=2).

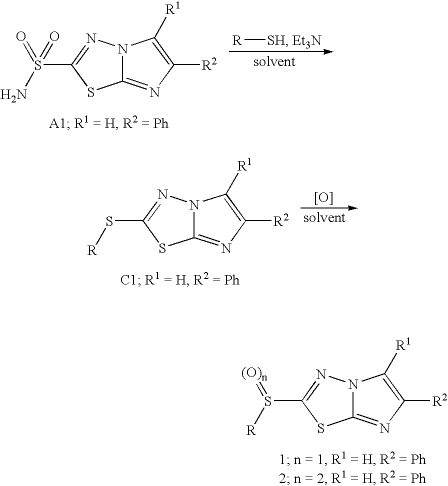

The selectivity of the oxidation may be controlled by the use of various oxidation conditions. For example, the use of 1 equiv of tert-BuOOH and silica gel in methylene chloride, or the use of 1 equiv of peracetic acid provide the sulfoxide as the major product. The addition of 2-3 equiv of oxidant, or the use of m-CPBA provides the sulfone. Sulfone and sulfoxide are readily separable by silica gel chromatography or recrystallization from an appropriate solvent.

Intermediate A is prepared as previously reported (PCT Application No. CA02/01942 (WO 03/051890)) by the condensation of 2-amino-1,3,4-thiadiazole-5-sulfonamide with the appropriate α-bromoketone.

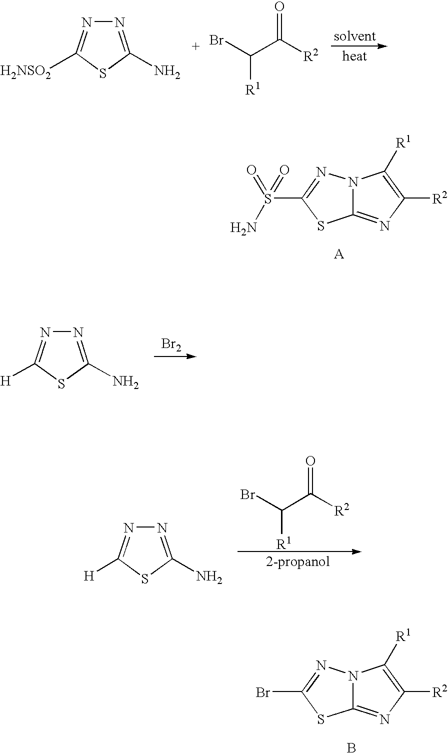

Intermediate B is prepared by the bromination of 2-amino-1,3,4-thiadiazole to 2-amino-5-bromo-1,3,4-thiadiazole and subsequent condensation with the appropriate α-bromoketone.

The following section provides selected variations on this procedure and subsequent functionalization of the sulfoxides and sulfones so obtained.

In the following example Boc(H)NCH₂CH₂SH is used as a nucleophile allowing for the preparation of amino functionalized sulfoxide and sulfone derivatives. Treatment of A1 with Boc(H)NCH₂CH₂SH under basic conditions yields intermediate C-43, which is oxidized to the sulfone, compounds 43.

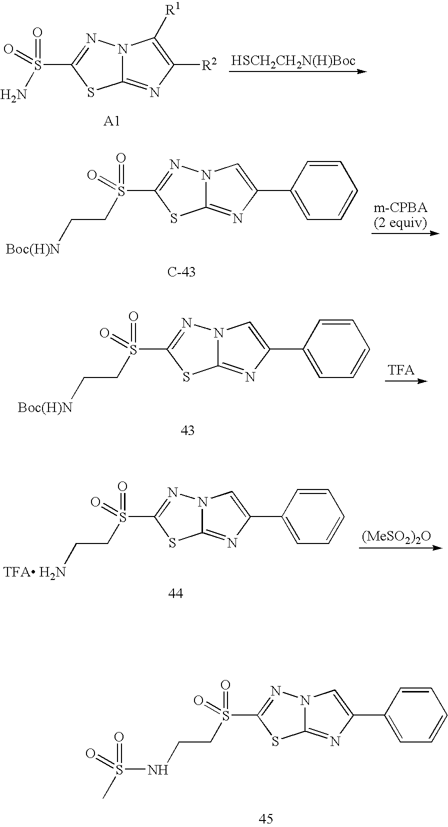

Deprotection of the Boc group with TFA provides the corresponding TFA.amine salt 44. Further functionalization of the TFA.amine salt 44 with methanesulfonic anhydride provides the sulfonamide, compound 45. In a similar fashion, functionalization with acid chlorides, anhydrides or coupling of carboxylic acids provide a variety of amides.

This functionalization may be done at an earlier step as illustrated below.

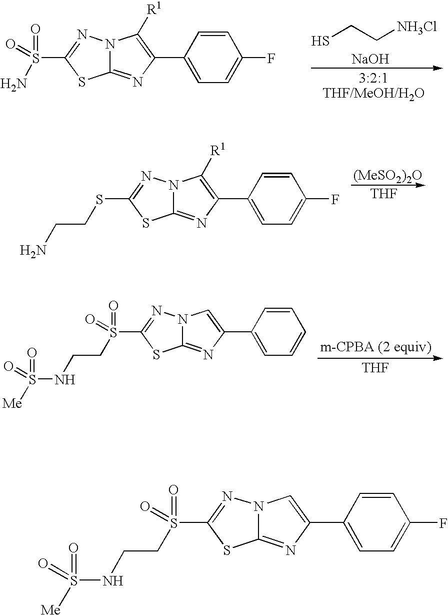

This chemistry has been extended to the preparation of photo affinity and biotinylated molecular probes such as compound 53, as illustrated below. 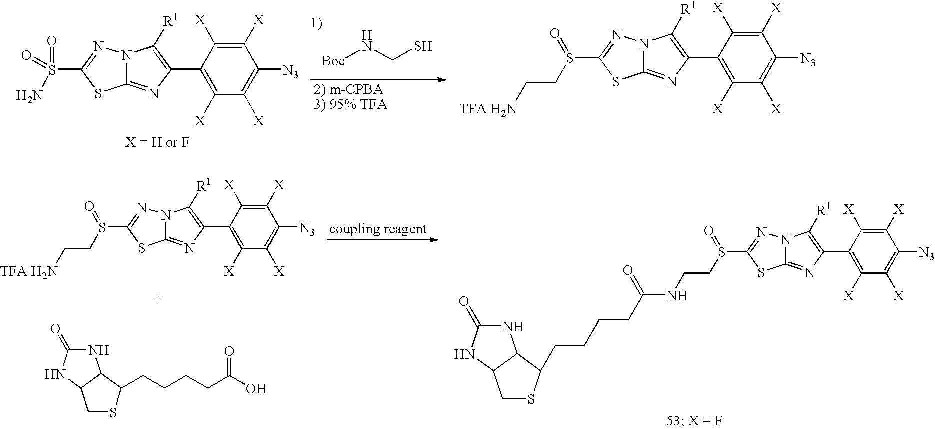
General Procedure B:

An alternate route to the sulfones is provided by sulfinate displacement of the bromine from intermediate B, to provide the desired sulfones represented by formula I.

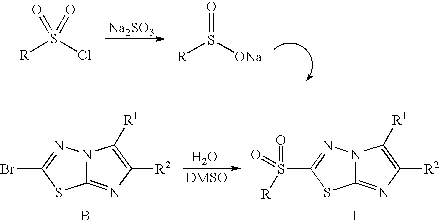

The requisite sodium sulfinate may be prepared by treating the appropriate sulfonyl chloride with Na₂SO₃ in water. This solution is then added to a DMSO/water solution of the appropriate intermediate B, to provide direct access to the sulfone represented by formula I.

General Procedure C:

A third route to sulfoxides and sulfones bearing a basic moiety on the left hand side of the molecule is illustrated below.

Compound A1 is treated with NaSH in MeOH to provide intermediate thiol D1 in near quantitative yield. D1 is alkylated with dibromoethane to provide intermediate sulfide E1. Oxidation with 1 equiv of m-CPBA yields the sulfoxide intermediate F1.

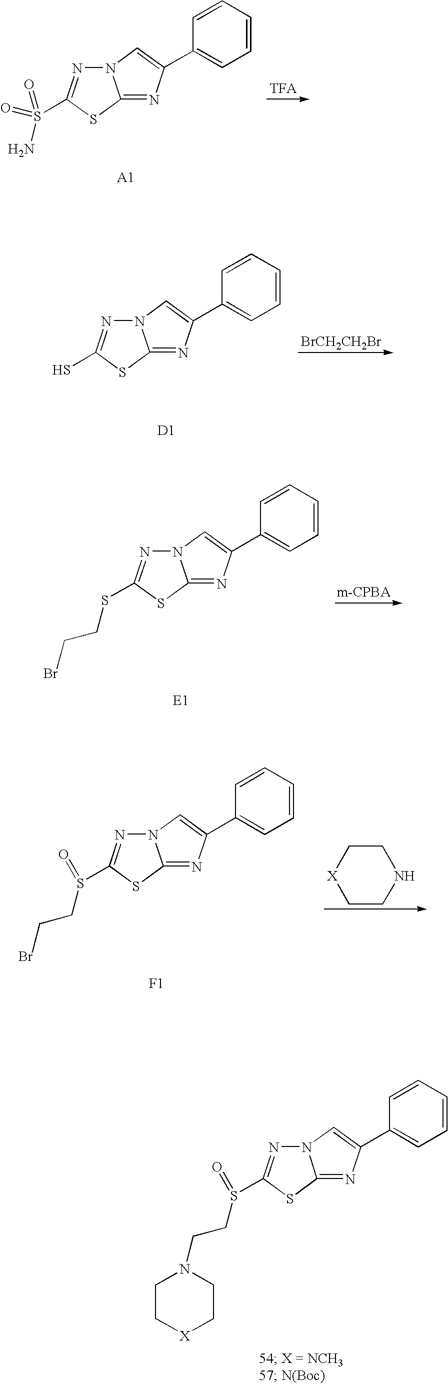

Intermediate F1 is then treated with a nucleophile such as morpholine or N-methylpiperazine to provide compounds 54 and 55, respectively. Other nucleophilic amines such as N-acylpiperazine, N-Boc-piperazine, N-methylhomopiperazine, N-Boc-homopiperazine, N-acetylhomopiperazine, 4-hydroxypiperidine, and imidazole provide related compounds.

Deprotection of compound 57 with neat TFA or another acid provides access to the secondary amines such as compound 58.

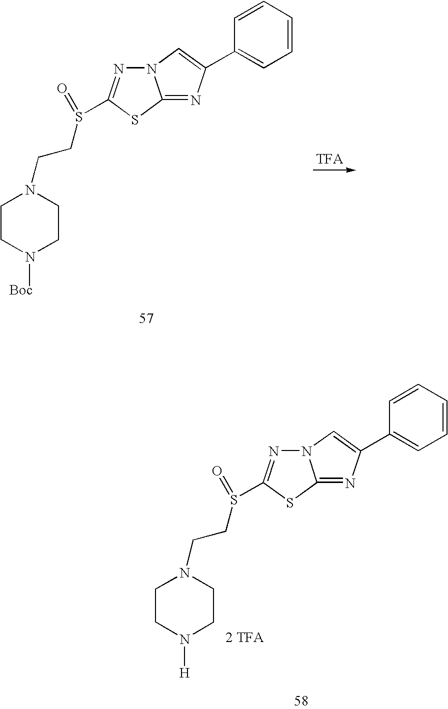

### EXAMPLES

General Procedure A: Sulfide Formation and Oxidation.

Step 1: Intermediate C.

Intermediate C may be prepared by treating the appropriate 6-arylimidazolo[2,1-b]-1,3,4 thiadiazole sulfonamide (intermediate A, 1 equiv) or 2-bromo-6-arylimidazolo[2,1-b]-1,3,4-thiadiazole (intermediate B, 1 equiv) with the appropriate thiol (1 equiv) and triethylamine (2-3 equiv) in methanol. The resulting solutions is stirred at room temperature or refluxed until intermediate A or B has been consumed, as determined by TLC. The solution is cooled and if a solid forms the desired intermediate C is filtered and washed with methanol. Alternatively, volatiles are removed under reduced pressure and standard aqueous work-up and purification by recrystallization or silica gel chromatography yields the desired intermediate C.

Step 2: Sulfide Oxidation.

a) tert-BuOOH/Silica Gel Oxidation

Intermediate C (1 equiv) is dissolved in methylene chloride and treated with silica gel (1 g/mmol) and 1-3 equiv of t-BuOOH. The reaction mixture is stirred at the appropriate temperature until intermediate C is converted to the desired mixture of sulfoxide and sulfone. If no reaction is observed the solution may be refluxed and/or oxone (1-3 equiv) is added. Solids are removed by filtration, washing with methylene chloride and THF/MeOH (1:1). The crude mixture may be purified by recrystallization or silica gel chromatography.

b) Peracetic Acid Oxidation

Intermediate C (1 equiv) is dissolved in an appropriate solvent such as methylene chloride or THF, and peracetic acid (1-3 equiv) is added. The solution is stirred at room temperature for 1-16 hours. Volatiles are removed under reduced pressure to provide crude sulfoxide, which may be further purified by recrystallization or silica gel chromatography.

c) m-CPBA Oxidation

Intermediate C (1 equiv) is dissolved in THF and cooled to 4° C. Solid m-CPBA (2-3 equiv) is added in one portion and the resulting solution stirred for 16-24 hours. Ethyl acetate extraction, washing with saturated aqueous Na₂S₃O₈, saturated aqueous NaHCO₃, and brine, provides crude sulfone, which may be further purified by recrystallization or silica gel chromatography.

General Procedure B: Sulfinate Displacement Reaction.

The appropriate sulfonyl chloride (1 equiv) is suspended or dissolved in water and treated with Na₂SO₃ (1.5 equiv) and K₂CO₃ (2 equiv). The solution is stirred at room temperature for 1 hour. The appropriate intermediate C (1 equiv) is dissolved in 50% DMSO/water. The solutions are mixed and refluxed until intermediate C has been consumed, as determined by TLC. Water is added and the resulting solid is either filtered off or extracted with ethyl acetate to provide the crude sulfone, which may be further purified by recrystallization from an appropriate solvent system or purified by silica gel chromatography.

General Procedure C:

Step 1: Intermediate D

To a stirred suspension of A in MeOH is added sodium hydrosulfide hydrate (3 equivalents) at room temperature. The mixture is stirred for one hour before volatiles are removed in vacuo. To the solid is added DCM with a small amount of MeOH to make a suspension, which is filtered and the solid washed with DCM. The filtrate is collected and the volatiles are removed in-vacuo to affording intermediate D.

Step 2: Intermediate E

Intermediate D is added to a solution of 1,2-dibromoethane (10 equivalents) in THF and stirred at room temperature for 16 hours. Volatiles are removed in-vacuo and the resulting oil/solid is adsorbed onto silica gel and purified by flash silica gel chromatography.

Step 3: Intermediate F

Intermediate E is dissolved in CHCl₃ and chilled to −14° C., then with swirling, m-CPBA (1.04 equiv) is added to the solution and the mixture is left at −14° C. for 16 hours. The organic layer is washed with water, dried over anhydrous MgSO₄, filtered and volatiles removed under reduced pressure. The resulting solid is purified by silica gel chromatography to provide he desired sulfoxide and/or sulfone.
Compounds 1 and 2:

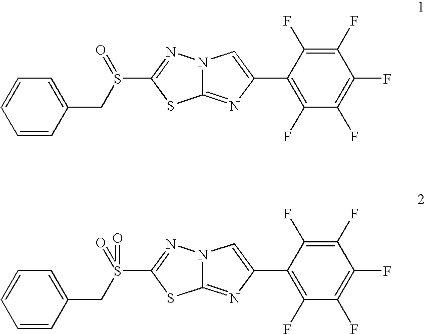

Compounds 1 and 2 were prepared according to General Procedure A.

Compound 1: ¹H NMR (200 MHz, CDCl₃) δ 8.15 (s, 1H), 7.38 (m, 3H), 7.24 (d, 2H), 4.46 (m, 2H).

Compound 2: ¹H NMR (200 MHz, CDCl₃) δ 8.12 (s, 1H), 7.35 (m, 5H), 4.74 (s, 2H).
Compounds 3 and 4:

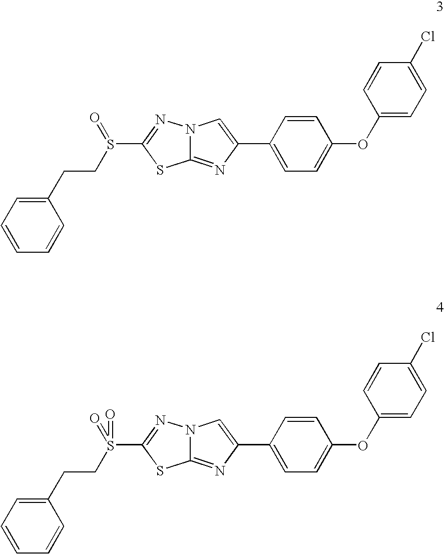

Compounds 3 and 4 were prepared according to General Procedure A.

Compound 3: ¹H NMR (200 MHz, CDCl₃) δ 3.10-3.90 (m, 2H), 3.47-3.55 (t, J=8.2 Hz, 2H), 6.95-7.07 (m, 4H), 7.22-7.33 (m, 7H), 7.78-7.82 (d, J=8.6 Hz, 2H), 7.97 (s, 1H). ¹³C NMR (CDCl₃, 50 MHz) δ 52.8, 83.2, 134.1, 144.4, 145.5, 152.2, 152.5, 154.0, 155.1, 162.6, 171.0, 172.0, 180.9, 182.3, 196.1. LCMS (M+1)=480.1.

Compounds 4: ¹H NMR (200 MHz, CDCl₃) δ 3.21-3.29 (t, J=8.2 Hz, 2H), 3.76-3.84 (t, J=8.6 Hz, 2H), 6.97-7.34 (m, 11H), 7.79-7.83 (d, J=8.9 Hz) 2H, 8.03 (s, 1H). ¹³C NMR (DMSO-d⁶, 50 MHz) δ 52.9, 80.7, 135.8, 144.2, 145.6, 151.8, 152.0, 153.4, 155.1, 162.1, 171.8, 180.6, 181.4, 185.4. LCMS (M+1)=496.1.
Compounds 5 and 6:

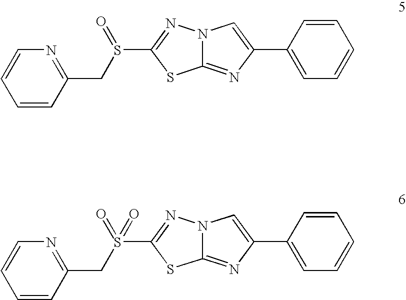

Compounds 5 and 6 were prepared according to General Procedure A.

Compound 5: ¹H NMR (200 MHz, CDCl₃) δ 8.51 (d, J=4.3 Hz, 1H), 8.03 (s, 1H), 7.81 (d, J=7.3 Hz, 2H), 7.70 (t of d, ¹J=7.6 Hz, ²J=1.2 Hz, 1H), 7.34 (m, 5H), 4.60 (m, 2H). ¹³C NMR (200 MHz, CDCl₃) δ 150.32, 148.47, 147.25, 145.71, 136.97, 133.17, 128.83, 128.104, 125.77, 125.28, 123.73, 109.29, 64.59.

Compound 6: ¹H NMR (200 MHz, CDCl₃) δ 8.43 (d, J=4.9 Hz, 1H), 8.13 (s, 1H), 7.85 (d of d, ¹J=8.6 Hz, ²J=1.8 Hz, 2H), 7.77 (t of d, ¹J=7.6 Hz, ²J=1.8 Hz, 1H), 7.43 (m, 5H), 4.90 (s, 2H). ¹³C NMR (200 MHz, CDCl₃) δ 150.32, 137.48, 132.87, 128.99, 128.62, 126.19, 125.57, 124.28, 109.78, 64.22
Compounds 7 and 8:

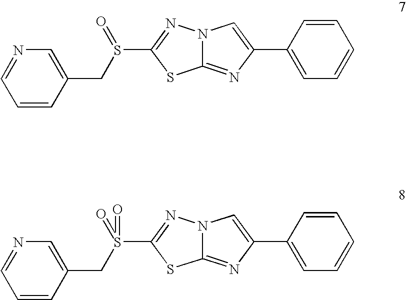

Compounds 7 and 8 were prepared according to General Procedure A.

Compound 7: ¹H NMR (200 MHz, CDCl₃) δ 8.94 (s, 1H), 8.58 (d, J=4.6 Hz, 1H), 8.52 (s, 1H), 7.92 (d, J=7.9 Hz, 2H), 7.81 (d, J=7.9 Hz, 1H), 7.44 (m, 4H), 5.26 (s, 2H).
Compounds 9 and 10:

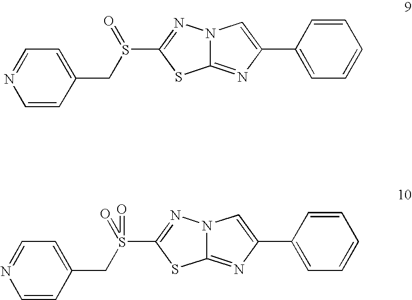

Compounds 9 and 10 were prepared according to General Procedure A.

Compound 9: ¹H NMR (200 MHz, CDCl₃) δ 8.61 (d, J=6.1 Hz, 2H), 8.05 (s, 1H), 7.83 (d, J=7.0 Hz, 2H), 7.39 (m, 3H), 7.16 (d, J=6.1 Hz, 2H), 4.40 (m, 2H).

Compound 10:
Compounds 11 and 12:

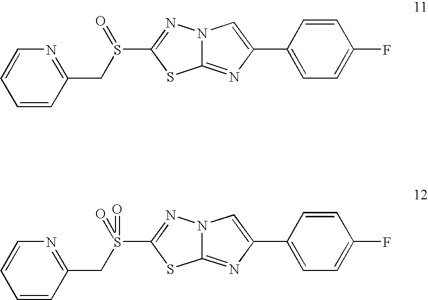

Compounds 11 and 12 were prepared according to General Procedure A.

Compound 11: ¹H NMR (200 MHz, DMSO-d⁶) δ 8.78 (s, 1H), 8.51 (d, J=5.5 Hz, 2H), 7.91 (dd, J=8.6 Hz, J=5.5 Hz, 2H), 7.26 (m, 4H), 4.72 (m, 2H).

Compound 12: ¹H NMR (200 MHz, DMSO-d⁶) δ 8.93 (s, 1H), 8.58 (d, J=4.0 Hz, 2H), 7.94 (t, J=7.9 Hz, 2H), 7.38 (d, J=4.6 Hz, 2H), 7.28 (t, J=8.9 Hz, 2H), 5.27 (s, 2H).
Compounds 13 and 14:

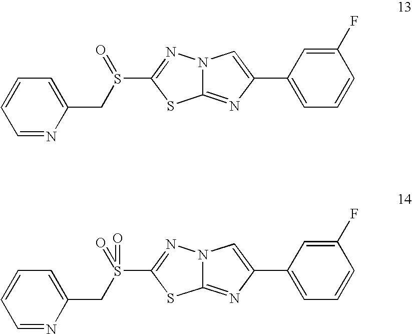

Compounds 13 and 14 were prepared according to General Procedure A.

Compound 13: ¹H NMR (200 MHz, DMSO-d⁶) δ 8.89 (s, 1H), 8.46 (dd, ¹J=4.9 Hz, ²J=0.9 Hz, 1H), 7.81 (t, J=7.6 Hz, 1H), 7.71 (t, J=12.2 Hz, 2H), 7.44 (m, 3H), 7.13 (t, J=7.3 Hz, 1H), 4.82 (m, 2H).

Compound 14: ¹H NMR (200 MHz, DMSO-d⁶) δ 9.04 (s, 1H), 8.41 (d, J=4.9 Hz, 1H), 7.86 (t, J=7.6 Hz, 1H), 7.75 (t, J=7.6 Hz, 2H), 7.41 (m, 3H), 7.16 (t, J=8.6 Hz, 1H), 5.30 (s, 2H).
Compounds 15 and 16:

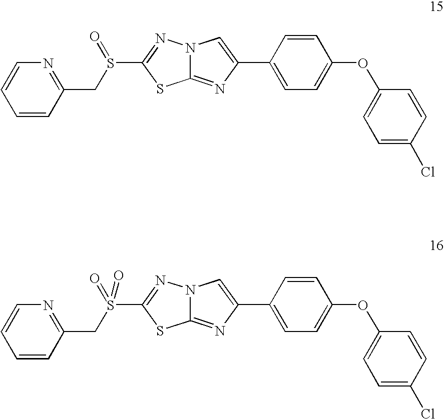

Compounds 15 and 16 were prepared according to General Procedure A

Compound 15: ¹H NMR (200 MHz, DMSO-d⁶) δ 8.74 (s, 1H), 8.46 (d, J=4.6 Hz, 1H), 7.90 (d, J=7.9 Hz, 2H), 7.82 (t, J=7.6 Hz, 1H), 7.40 (m, 4H), 7.07 (m, 4H), 4.81 (m, 2H).

Compound 16: ¹H NMR (200 MHz, DMSO-d⁶) δ 8.91 (s, 1H), 8.42 (d, J=4.9 Hz, 1H), 7.94 (d, J=8.6 Hz, 2H), 7.86 (t, J=7.6 Hz, 1H), 7.55 (d, J=7.6 Hz, 1H), 7.42 (m, 3H), 7.09 (m, 4H), 5.29 (s, 2H).
Compounds 17 and 18:

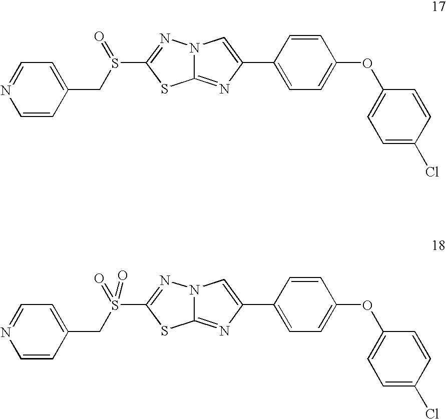

Compounds 17 and 18 were prepared according to General Procedure A

Compound 17: ¹H NMR (200 MHz, DMSO-d⁶) δ 8.76 (s, 1H), 8.51 (d, J=5.2 Hz, 2H), 7.90 (d, J=8.2 Hz, 2H), 7.43 (d, J=8.6 Hz, 2H), 7.25 (d, J=5.5 Hz, 2H), 7.07 (m, 4H), 4.72 (m, 2H).

Compound 18: ¹H NMR (200 MHz, DMSO-d⁶) δ 8.91 (s, 1H), 8.86 (d, J=5.8 Hz, 2H), 7.93 (d, J=8.6 Hz, 2H), 7.44 (m, 2H), 7.39 (d, J=6.1 Hz, 2H), 7.09 (m, 4H), 5.27 (s, 2H).
Compounds 19 and 20:

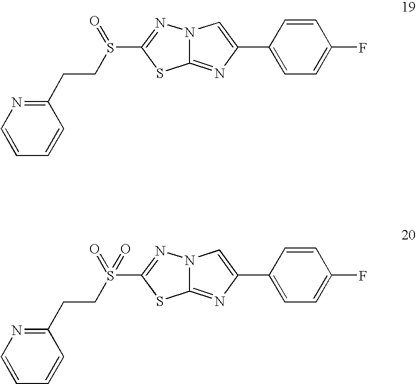

Compounds 19 and 20 were prepared according to General Procedure A.

Compound 19: ¹H NMR (CDCl₃, 200 MHz) δ 3.31-3.50 (m, 2H), 3.34-3.88 (m, 2H), 7.07-7.26 (m, 4H), 7.62 (dt, 1H, J=1.6 Hz, J=7.2 Hz), 7.76-7.83 (m, 2H), 7.96 (s, 1H), 8.49 (d, J=5.9 Hz). LCMS (M+1)=372.5.

Compound 20: ¹H NMR (CDCl₃, 200 MHz) δ 3.41 (t, 2H, J=7.5 Hz), 4.07 (t, 2H, J=7.4 Hz), 7.10-7.22 (m, 4H), 7.61 (m, 2H), 7.79 (m, 2H), 8.00 (s, 1H), 8.39 (d, J=4.5 Hz). LCMS (M+1)=388.5.
Compound 21:

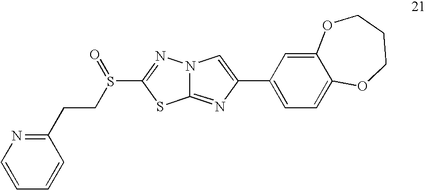

Compound 21 was prepared according to General Procedure A. ¹H NMR (CDCl₃, 200 MHz) δ 2.21 (m, 2H), 3.20-3.43 (m, 2H), 3.61-3.83 (m, 2H), 4.25 (t, 4H, J=7.4 Hz), 7.01 (d, 1H, J=8.6 Hz), 7.05-7.21 (m, 2H), 7.30-7.42 (m, 2H), 7.59 (t, 1Hm J=7.5 Hz), 7.88 (s, 1H), 8.48 (d, 1H, J=5.8 Hz). LCMS (M+1)=426.5.
Compound 23:

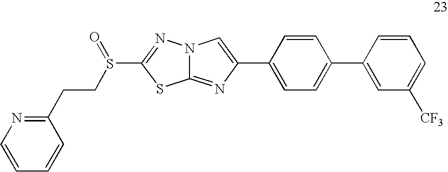

Compound 23 was prepared according to General Procedure A. ¹H NMR (200 MHz, CDCl₃) δ 3.30-3.45 (m, 2H), 3.74-3.85 (m, 2H), 7.11 (m, 2H), 7.56-7.69 (m, 6H), 7.80-7.96 (m, 5H), 8.08 (s, 1H), 8.49 (d, 1H, J=5.8 Hz). LCMS (M+1)=498.6.
Compound 24:

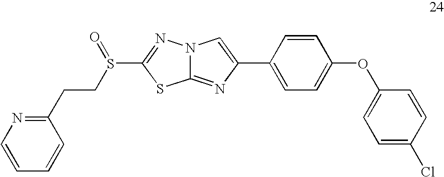

Compound 24 was prepared according to General Procedure A. ¹H NMR (200 MHz, CDCl₃) δ 3.29-3.48 (m, 2H), 3.72-3.79 (m, 2H), 6.95-7.11 (m, 4H), 7.15-7.31 (m, 4H), 7.61 (t, 1H, 7.6 Hz), 7.70 (d, 2H, J=7.6 Hz), 7.96 (s, 1H), 8.50 (d, 1H, J=4.4 Hz). LCMS (M+1)=481.0.
Compound 25 and 26:

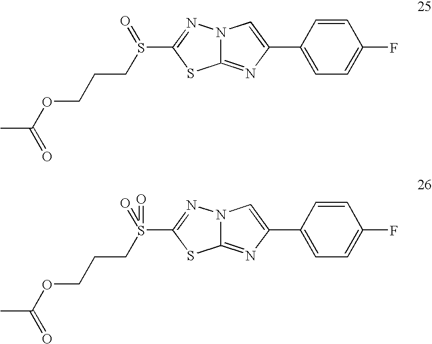

Compounds 25 and 26 were prepared according to General Procedure A

Compound 25: ¹H NMR (200 MHz, DMSO-d⁶) δ 8.80 (s, 1H), 7.92 (dd, ¹J=9.2 Hz, ²J=5.5 Hz, 2H), 7.26 (t, J=8.9 Hz, 2H), 4.10 (t, J=6.1 Hz, 2H), 3.40 (m, 2H), 2.00 (m, 2H), 1.98 (s, 3H).

Compound 26: ¹H NMR (200 MHz, DMSO-d⁶) δ 8.93 (s, 1H), 7.95 (dd, ¹J=8.9 Hz, ²J=5.8 Hz, 2H), 7.28 (t, J=9.2 Hz, 2H), 4.07 (t, J=6.4 Hz, 2H), 3.82 (t, J=7.6 Hz, 2H), 2.09 (m, 2H), 1.98 (s, 3H).
Compound 27:

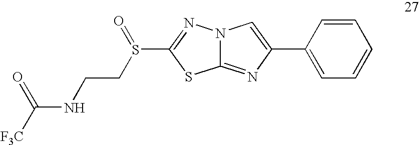

¹H NMR (200 MHz, CDCl₃), δ 8.07 (s, 1H), 7.84 (d, J=7.6 Hz, 2H), 7.45 (t, J=7.6 Hz, 2H), 7.37 (m, 1H), 4.00 (m, 2H), 3.61 (m, 2H).
Compound 28:

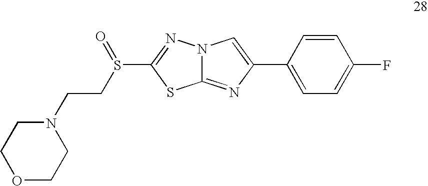

Compound 28 was prepared according to General Procedure A. ¹H NMR (200 MHz, CDCl₃)δ 7.96 (s, 1H), 7.78 (m, 2H), 7.11 (m, 2H), 3.53 (m, 4H) 3.38 (tJ=6.2 Hz, 2H), 2.93 (q, J=6.2 Hz, 2H), 2.496 (m, 4H).
Compound 29:

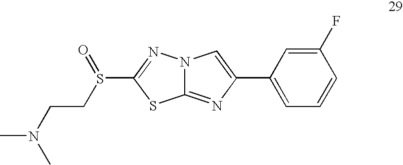

Compound 29 was prepared according to General Procedure A. ¹H NMR (CDCl₃, 200 MHz) δ 2.31 (s, 6H), 2.74-3.00 (m, 2H), 3.36 (t, J=6.4 Hz, 2H), 6.97-7.06 (dt, J=8.6 Hz, J=2.4 Hz, 1H), 7.37 (dq, J=8.0 Hz, J=2.0 Hz, 1H), 7.57 (t, J=7.6 Hz, 2H), 8.03 (s, 1H). ¹³C NMR (CDCl₃, 50 MHz) δ69.3, 75.3, 80.0, 134.0, 136.5, 136.9, 139.0, 139.5, 145.3, 154.8, 170.5, 190.2. LCMS (M+H)=329.1.
Compound 30:

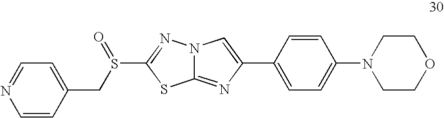

Compound 30 was prepared according to General Procedure A. ¹H NMR (200 MHz, CDCl₃), δ 8.44 (d, 2H, J=5.0 Hz), 7.91 (s, 1H), 7.59 (d, 2H, J=8.6 Hz), 7.21 (d, 1H, J=5.0 Hz), 6.88 (d, 2H, J=8.6 Hz), 4.42 (m, 2H), 3.77 (m, 4H), 3.11 (m, 4H).
Compound 31:

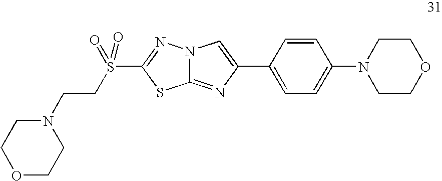

Compound 31 was prepared according to General Procedure A. ¹H NMR (200 MHz, CDCl₃) δ 7.86 (s, 1H), 7.70 (d, J=9.00 Hz, 2H), 6.95 (d, J=8.60 Hz, 2H), 4.43 (m, 2H), 3.85 (m, 6H), 3.401 (m, 4H), 3.20(m, 4H), 2.52 (m, 4H).
Compound 32 and 33:

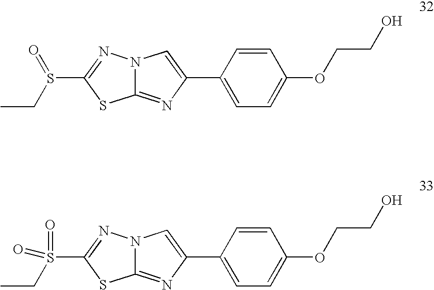

Compounds 32 and 33 were prepared according to General Procedure A.

Compound 32: ¹H NMR (CDCl₃, 200 MHz) δ 1.42 (t, 3H, J=7.6 Hz), 2.50, (br s, 1H), 3.19-3.31 (m, 2H), 3.98 (t, 2H, J=4.6 Hz), 4.14 (t, 2H, J=3.6 Hz). 6.98 (d, 2H, J=8.8 Hz), 7.76 (d, 2H, J=9.0 Hz). LCMS (M+1)=337.4.

Compound 33: ¹H NMR (CDCl₃, 200 MHz) δ 1.50 (m, 3H), 2.20 (br s, 1H), 3.47 (q, 2H, J=8.0 Hz), 3.99 (b, 2H), 4.14 (b, 2H), 6.99 (d, 2H, J=8.2 Hz), 7.70 (d, 2H, J=8.4 Hz), 8.05 (s, 1H). LCMS (M+1)=353.4.
Compound 34:

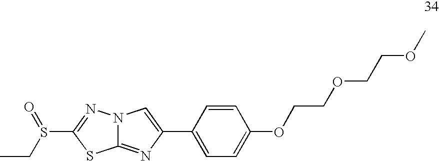

Compound 34 was prepared according to General Procedure A.

¹H NMR (CDCl₃, 200 MHz) δ1.40 (t, 3H, J=7.2 Hz), 3.21 (m, 2H), 3.38 (s, 3H), 3.57 (t, 2H, J=3.0 Hz), 3.73 (t, 3H, 4.2 Hz), 3.68 (J=4.2 Hz), 4.15 (t, 2H, J=4.0 Hz), 6.96 (d, 2H, J=8.6 Hz), 7.73 (d, 2H, J=8.6 Hz), 7.93 (s, 1H). LCMS (M+1)=395.5.
Compound 35:

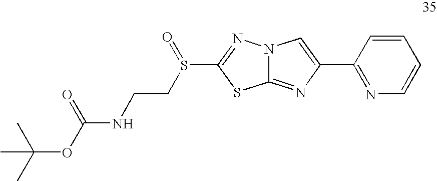

Compound 34 was prepared according to General Procedure A.

¹H NMR (CDCl₃, 200 MHz) δ 1.36 (s, 9H), 3.43-3.53 (m, 2H), 3.65-3.71 (m, 2H), 5.14 (br s, 1H), 7.16-7.25 (m, 1H), 7.70-7.78 (dt, 1H, J=1.6 Hz, J=7.6 Hz), 8.00 (d, 1H J=7.6 Hz), 8.40 (s, 1H), 8.56 (d, 1H, J=4.6 Hz). LCMS (M+1)=393.5.
Compound 36:

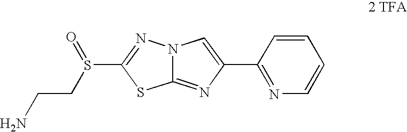

Compound 35 (500 mg) was stirred in wet THF (5 mL) for 10 minutes. Volatiles were removed under reduced pressure. Diethyl ether (10 mL) was added and the resulting white solid was filtered and washed with diethyl ether (3×10 mL) to provide compound 36 as a white solid (97% yield). ¹H NMR (200 MHz, CDCl₃) δ 3.40-3.81 (m, 4H), 7.78 (t, 1H, J=7.4 Hz), 8.24 (d, 1H, J=7.8 Hz), 8.49 (t, 1H, J=8.0 Hz), 8.55 (d, 1H, 5.8 Hz), 8.83 (s, 1H). LCMS (M+1)=293.4.
Compound 37:

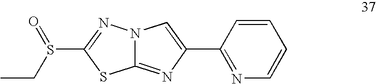

Compound 37 was prepared according to General Procedure A.

¹H NMR (200 MHz, CDCl₃) δ 1.42 (t, J=7.6 Hz, 3H), 3.28 (m, 2H), 7.21 (m, 1H), 7.76 (d, J=7.9 Hz, 1H), 8.00 (d, J=7.3 Hz), 8.41 (s, 1H), 8.59 (d, J=4.9 Hz). ¹³C NMR (50 MHz, CDCl₃) δ 30.0, 75.0, 136.5, 144.2, 147.2, 161.3, 174.0. LCMS (M+H)=279.0.
Compound 38:

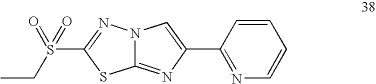

Compound 38 was prepared according to General Procedure A.

¹H NMR (200 MHz, CDCl₃) δ 1.51 (t, 3H, J=7.4 Hz), 3.54 (q, 2H, J=7.4 Hz), 7.28 (m, 1H), 7.84 (t, 1H, J=6.4 Hz), 8.08 (d, 1H, J=7.8 Hz), 8.63 (br, 1H). LCMS (M+1) 294.4.
Compound 39:

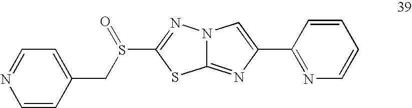

Compound 39 was prepared according to General Procedure A.

¹H NMR (200 MHz, CDCl₃) δ 8.60 (m, 3H), 8.44 (s, 1H), 8.02 (d, J=8.1 Hz, 2H), 7.78 (t, J=8.1 Hz, 1H), 7.15 (d, J=4.6 Hz, 2H), 4.42 (dd, J₁=16.8 Hz, J₂=13.2 Hz, 2H).
Compound 40:

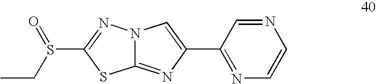

Compound 40 was prepared according to General Procedure A.

¹H NMR (200 MHz, CDCl₃) δ 9.29 (s, 1H), 8.52 (m, 2H), 8.44 (s, 1H), 3.30 (m, 2H), 1.44 (t, J=7.2 Hz, 3H).
Compounds 41 and 42:

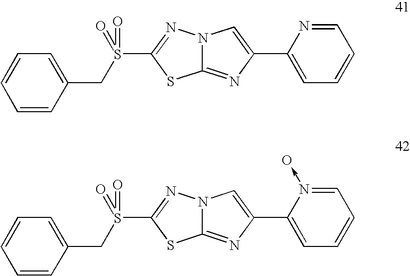

Compounds 41 and 42 were prepared according to General Procedure A.

Compound 41: ¹H NMR (200 MHz, CDCl₃), δ 8.62 (d, 1H), 8.56 (s, 1H), 8.05 (d, 1H), 7.79 (t 1H), 7.33 (m, 6H), 4.73 (s, 1H).

Compound 42: ¹H NMR (200 MHz, CDCl₃), δ 9.50 (s, 1H), 8.36 (m, 2H), 7.33 (m, 7H), 4.75 (s 2H).
Compound 43:

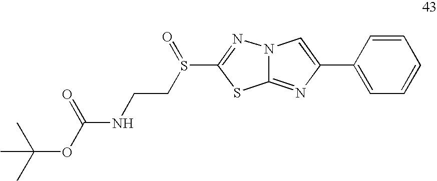

Compound 43 was prepared according to General Procedure A-c.

¹H NMR (200 MHz, DMSO-d⁶) δ 8.95 (s, 1H), 7.91 (d, 2H), 7.44 (t, 2H), 7.36 (t, 1H), 6.98 (t, 1H), 3.84 (t, 2H), 3.442 (t, 2H), 1.13 (s, 9H).
Compound 44:

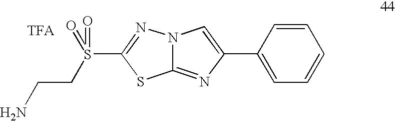

Compound 43 (2.5 g) was stirred in 95% wet TFA until all solid had dissolved. Volatiles were removed under reduced pressure and the resulting oil treated with diethyl ether. The resulting solid was filtered and washed with diethyl ether (3×10 mL) to provide compound 44 as a white solid (96% yield).

¹H NMR (200 MHz, DMSO-d⁶) δ 8.95 (s, 1H), 8.10 (br s, 2H), 7.85 (d, 2H), 7.42 (t, 2H), 7.31 (t, 1H), 4.02 (t, 2H), 3.31 (br s, 2H).
Compound 45:

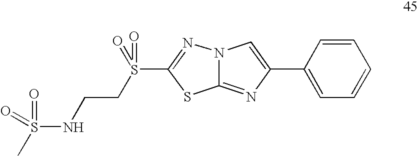

Compound 44 (2.5 g) was dissolved in THF (5 mL) and treated with triethylamine, DMAP (2 mg), and methanesulonic anhydride( ). The solution was stirred at room temperature for 16 hours. Standard ethyl acetate/water work-up provided the desired compounds 45 as a white solid 85% yield. LCMS (API-ES, pos scan, m/z) M+1=387.1.
Compound 46:

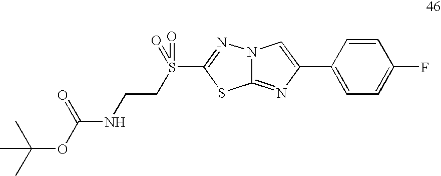

Compound 46 was prepared according to General Procedure A-c and recrystallized from MeOH. ¹H NMR (200 MHz, DMSO-d⁶) δ 8.91 (s, 1H), 7.94 (dd, J=5.5, 8.8 Hz, 2H), 7.24 (t, J=8.8 Hz, 2H), 6.97 (br t, 1H), 3.87 (t, J=6.1 Hz, 2H), 3.42 (t, J=6.1 Hz, 2H), 1.24 (s, 9H).
Compound 47:

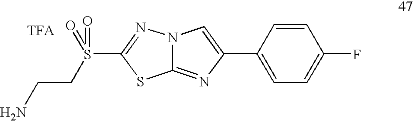

Compound 47 was prepared as per compound 44. ¹H NMR (200 MHz, DMSO-d⁶) δ 8.94 (s, 1H), 8.10 (br s, 2H), 7.92 (t, 2H), 7.28 (t, 2H), 4.07 (t, 2H), 3.38 (t, 2H).
Compound 48:

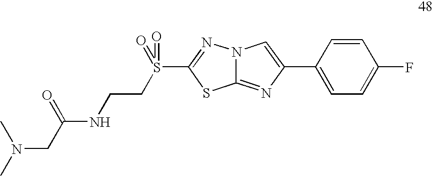

Compound 47 was refluxed in THF with N-N-dimethylglycine (1 equiv), 2-chloromethylpyridinium hydrochloride (2 equiv) and triethylamine (3 equiv) for 16 hours. Standard ethyl acetate/water work-up provide compound 48 which was further purified by silica gel chromatography, eluting with 10:1 methylene chloride/methanol, to provide compound 48 as a white solid in 13% yield. LCMS (API-ES, pos scan, m/z) M+1=412.1.
Compound 49:

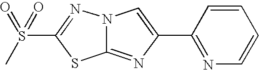

Compound 49 was prepared according to General Procedure B.

¹H NMR (200 MHz, DMSO-d⁶) δ 8.97 (s, 1H), 8.62 (d, J=4.9 Hz, 1H), 8.06 (m, 2H), 7.42 (t, J=5.8 Hz, 1H), 3.67 (s, 3H).
Compound 50:

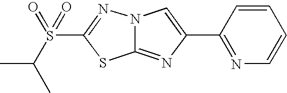

Compound 50 was prepared according to General Procedure B.

¹H NMR (200 MHz, DMSO-d⁶) δ 8.60 (d, J=4.0 Hz, 1H), 8.54 (s, 1H), 8.04 (d, J=7.3 Hz, 1H), 7.79 (t, J=7.6 Hz, 1H), 7.25 (t, J=4.9 Hz, 1H), 3.65 (septet, J=7.0 Hz, 1H), 1.51 (d, J=7.0 Hz, 6H).
Compound 51:

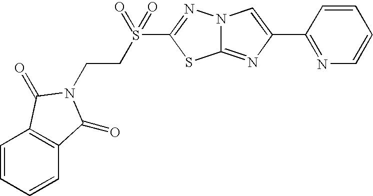

Compound 51 was prepared according to General Procedure B.

¹H NMR (200 MHz, DMSO-d⁶) δ 8.69 (s, 1H), 8.60 (d, J=5.1 Hz, 1H), 7.79 (m, 6H), 7.34 (t, J=5.5 Hz, 1H), 4.17 (m, 4H).
Compound 52:

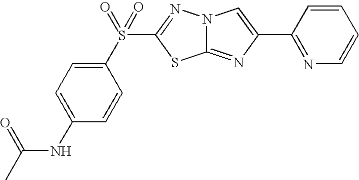

Compound 52 was prepared according to General Procedure B.

¹H NMR (200 MHz, DMSO-d⁶) δ 10.56 (s, 1H), 8.78 (s, 1H), 8.55 (d, J=5.8 Hz, 1H), 7.98 (m, 6H), 7.31 (t, J=5.8 Hz, 1H), 2.08 (s, 3H).
Compound 53:

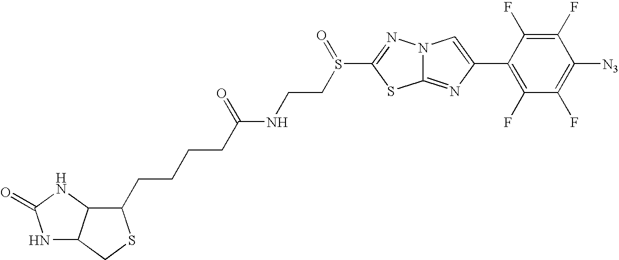

LC-MS (API, POS-scan, m/z): M+1=632

Compounds 54 to 71, and their respective intermediates are were prepared using General Procedure C as per compound 54, and are listed below.
Compound 54: 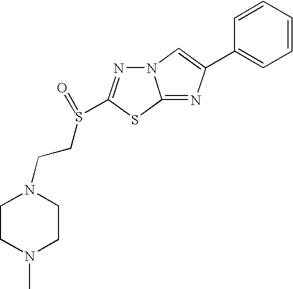
Intermediate D1:

To a stirred suspension of A1 in MeOH was added sodium hydrosulfide hydrate (3 equivalents) at room temperature. The mixture was stirred for one hour before volatiles were removed in vacuo. To the solid was added DCM with a small amount of MeOH to make a suspension, which was filtered and the solid washed with DCM. The filtrate was collected and the volatiles were removed in-vacuo to affording a yellow foam in quantitative yield. ¹H NMR (200 MHz, DMSO-d⁶) δ 8.01 (s, 1H), 7.72 (d, J=8.3 Hz, 2H), 7.30 (m, 2H), 7.13 (m, 1H).

Intermediate E1

Intermediate D1 was added to a solution of 1,2-Dibromoethane (10 equivalents) in THF and stirred at room temperature for 16 hours. Volatiles removed in-vacuo and resulting oil was adsorbed onto silica gel and purified by flash chromatography, eluting with 35% ethyl acetate/hexanes, to provide intermediate E1 as an off-white solid in 55-60% yield. ¹H NMR (200 MHz, CDCl₃) δ 7.98 (s, 1H), 7.80 (d, J=8.0 Hz, 2H), 7.42 (m, 3H), 3.69 (m, 4H).

Intermediate F1:

Intermediate E1 was dissolved in CHCl₃ and chilled to −14° C., then with swirling, m-CPBA (1.04 equivalents) was added to the solution and mixture left at −14° C. for 16 hours. The organic layer was washed with water, dried over anhydrous MgSO₄, filtered and volatiles removed under reduced pressure. The resulting solid was purified by silica gel chromatography, eluting with 35% ethyl acetate/hexanes, to provide a white solid in 70-80%. ¹H NMR (200 MHz, DMSO-d⁶) δ 8.83 (s, 1H), 7.89 (d, J=8.0 Hz, 2H), 7.42 (m, 3H), 3.87 (m, 4H).

Intermediate F1 was dissolved in dichloromethane. Piperidinomethylpolystyrene-HL (2 equivalents) was added to the stirred solution and then 1-Methylpiperazine (1.00 equivalents) was added. The mixture was stirred at room temperature for 16 hours, filtered and the resin washed with DCM/MeOH (1:1). The resulting filtrate was concentrated under reduced pressure and the resulting orange solid was dissolved in a minimum of THF. Diethyl ether and hexanes were added until crystallization initiated. The resulting solution was placed at −14° C. for 16 hours, the solid was filtered and washed with diethyl ether to provide beige crystals in 85% yield. ¹H NMR (200 MHz, CDCl₃) δ 8.02 (s, 1H), 7.83 (d, J=7.0 Hz, 2H), 7.43 (m, 3H), 3.36 (t, J=5.8 Hz, 2H), 2.93 (m, 2H), 2.53 (m, 4H), 2.29 (m, 4H), 2.16 (s, 3H).
Compound 56:

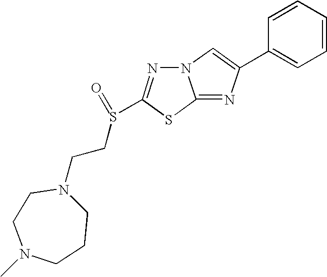

¹H NMR (200 MHz, CDCl₃) δ 8.03 (s, 1H), 7.82 (d, J=6.7 Hz, 2H), 7.43 (m, 3H), 3.36 (t, J=6.0 Hz, 2H), 3.14 (t, J=6.1 Hz, 2H), 2.88 (m, 8H), 2.52 (s, 3H), 2.03 (m, 2H).
Compound 57:

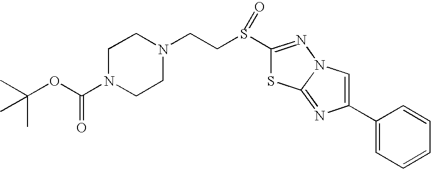

¹H NMR (200 MHz, CDCl₃) δ 8.03 (s, 1H), 7.83 (d, J=7.0 Hz, 2H), 7.43 (m, 3H), 3.38 (t, J=6.4 Hz, 2H), 3.30 (m, 4H), 2.94 (t, J=6.7 Hz, 2H), 2.45 (t, J=5.2 Hz, 4H), 1.42 (s, 9H).
Compound 58:

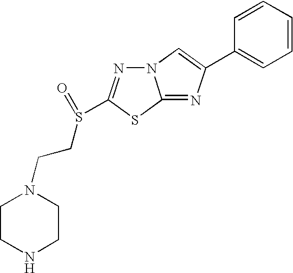

¹H NMR (200 MHz, CDCl₃) δ 8.04 (s, 1H), 7.84 (d, J=6.7 Hz, 2H); 7.44 (m, 3H), 3.38 (t, J=6.1 Hz, 2H), 7.97 (t, J=6.1 Hz, 2H), 2.89 (m, 4H), 2.62 (t, J=5.1 Hz, 4H).
Compound 59:

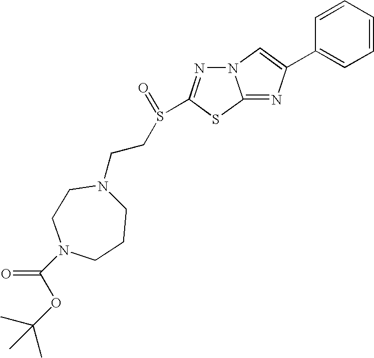

¹H NMR (200 MHz, CDCl₃) δ 8.02 (s, 1H), 7.83 (d, J=7.0 Hz, 2H), 7.43 (m, 3H), 3.35 (m, 6H), 3.07 (t, J=6.4 Hz, 2H), 2.72 (m, 4H), 1.77 (m, 2H), 1.44 (s, 9H).
Compound 60:

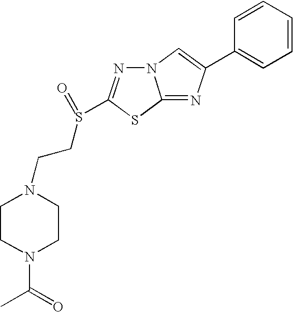

¹H NMR (200 MHz, CDCl₃) δ8.03 (s, 1H), 7.83 (d, J=7.4 Hz, 2H), 7.44 (m, 3H), 3.42 (m, 6H), 2.96 (m, 2H), 2.50 (m, 4H), 2.05 (s, 3H).
Compound 61:

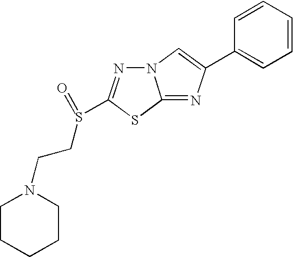

¹H NMR (200 MHz, CDCl₃) δ 8.03 (s, 1H), 7.83 (d, J=7.0 Hz, 2H), 7.43 (m, 3H), 3.37 (t, J=6.1 Hz, 2H), 2.86 (m, 2H), 2.42 (m, 4H), 1.38 (m, 6H).
Compound 62:

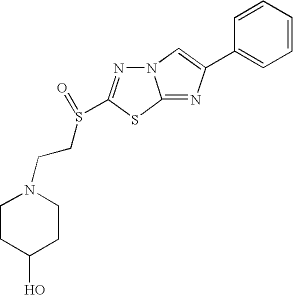

¹H NMR (200 MHz, CDCl₃) δ 8.01 (s, 1H), 7.80 (d, J=7.3 Hz, 2H), 7.40 (m, 3H), 3.63 (m, 1H), 3.35 (t, J=6.5 Hz, 2H), 2.88 (t, J=6.1 Hz, 2H), 2.75 (m, 2H), 2.23 (t, J=10.1 Hz, 2H), 1.47 (m, 2H), 1.40 (m, 2H).
Compound 63:

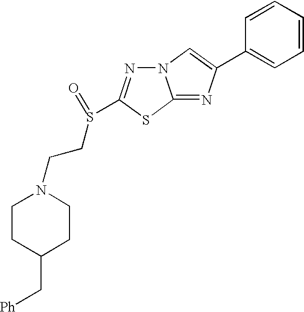

¹H NMR (200 MHz, CDCl₃) δ 7.75 (s, 1H), 7.58 (d, J=7.0 Hz, 2H), 7.17 (m, 3H), 6.94 (m, 5H), 3.05 (m, 4H), 2.63 (m, 2H), 2.20 (bs, 4H), 1.95 (bs, 4H).
Compound 64:

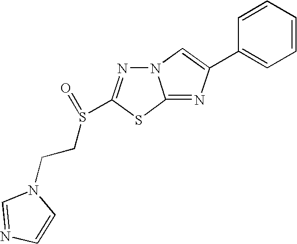

Compound 64 was prepared according to General Procedure C.

¹H NMR (200 MHz, DMSO-d⁶) δ 9.07 (s, 2H), 8.80 (s, 1H), 7.89 (d, J=7.3 Hz, 2H), 7.68 (m, 3H), 7.42 (m, 3H), 4.75 (m, 2H), 4.00 (m, 2H).
Intermediate A4:

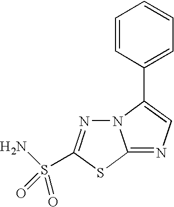

Intermediate A4 was prepared as previously described (Barnish, I. T., et al. J. Med. Chem., 23(2), 117-121, 1980). ¹H NMR (200 MHz, DMSO-d⁶) δ 8.80 (s, 1H), 7.98 (m, 2H), 7.51 (m, 3H).
Intermediate E4: General Procedure C.

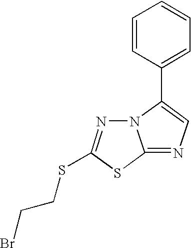

¹H NMR (200 MHz, CDCl₃) δ 7.89 (d, J=9.8 Hz, 2H), 7.55 (s, 1H), 7.46 (m, 3H), 3.74 (m, 4H).
Intermedaite F4: General Procedure C

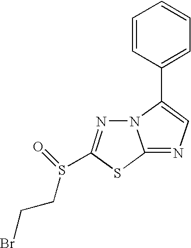

¹H NMR (200 MHz, CDCl₃) δ 7.89 (d, J=9.8 Hz, 2H), 7.55 (s, 1H), 7.46 (m, 3H), 3.74 (m, 4H).
Compound 65:

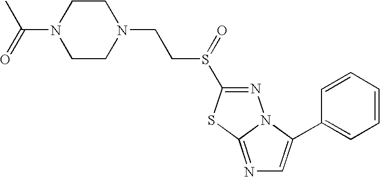

¹H NMR (200 MHz, CDCl₃) δ 7.84 (d, J=7.3 Hz, 7.63 (s, 1H), 7.46 (m, 3H), 3.35 (m, 6H), 2.96 (m, 2H), 2.46 (m, 2H), 2.03 (s, 3H).
Compound 66:

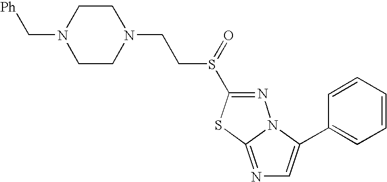

¹H NMR (200 MHz, DMSO-d⁶) δ 7.86 (d, J=7.0 Hz, 2H), 7.66 (s, 1H), 7.46 (m, 2H), 7.25 (m, 6H), 3.35 (m, 4H), 2.93 (m, 2H), 2.47 (m, 4H), 2.21 (m, 4H).
Compound 67:

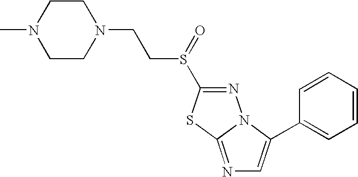

¹H NMR (200 MHz, CDCl₃) δ 7.85 (d, J=7.6 Hz, 2H), 7.64 (s, 1H), 7.46 (m, 3H), 3.40 (m, 2H), 2.95 (m, 2H), 2.50 (s, 4), 2.15 (s, 7H).
Intermediate E6:

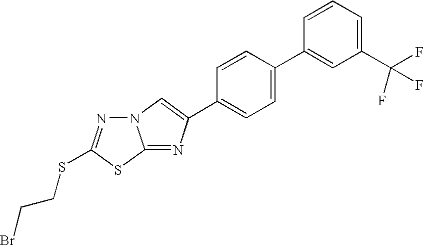

¹H NMR (200 MHz, CDCl₃) δ 8.05 (s, 1H), 7.89 (m, 3H), 7.84 (m, 1H), 7.64 (m, 4H), 3.71 (m, 4H).
Intermediate F6:

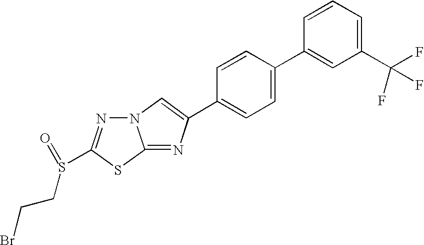

¹H NMR (200 MHz, CDCl₃)δ8.11 (s, 1H), 7.90 (m, 3H), 7.80 (d, J=6.7 Hz, 1HO, 7.63 (m, 4H), 3.80 (m, 2H), 3.72 (m, 2H).
Compound 68:

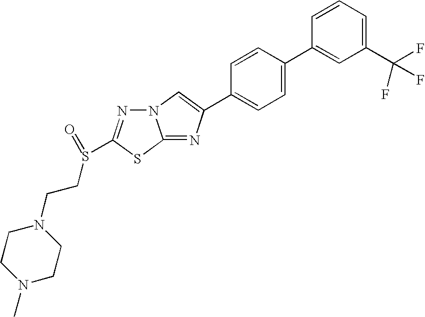

¹H NMR (200 MHz, DMSO-d⁶) δ 8.94 (s, 1H), 8.01 (m, 4H), 7.83 (m, 2H), 7.72 (m, 2H), 3.83 (m, 2H), 3.56 (m, 6H), 3.24 (m, 4H), 2.75 (s, 3H).
Intermediate E6: Prepared According to General Procedure C Using 1,3-dibromopropane in Place of 1,2-dibromoethane.

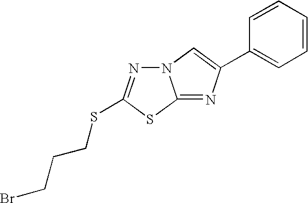

¹H NMR (200 MHz, CDCl₃) δ 7.96 (s, 1H), 7.80 (d, J=7.4 Hz, 2H), 7.41 (t, J=7.2 Hz, 2H), 7.31 (d, J=7.0 Hz, 1H), 3.57 (t, J=6.1 Hz, 2H), 3.42 (t, J=6.7 Hz, 2H), 2.37 (m, 2H).
Intermediate F6: General Procedure C

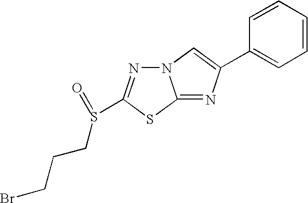

¹H NMR (200 MHz, CDCl₃) δ 8.07 (s, 1H), 7.84 (d, J=7.0 Hz, 2H), 7.44 (m, 3H), 3.56 (t, J=6.4 Hz, 2H), 3.43 (m, 2H), 2.44 (m, 2H).
Compound 69:

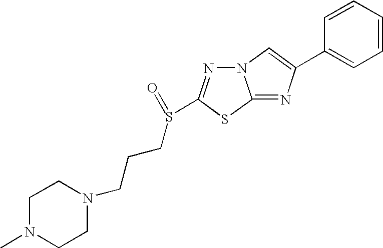

¹H NMR (200 MHz, CDCl₃) δ 8.03 (s, 1H), 7.82 (d, J=8.0 Hz, 2H), 7.42 (m, 3H), 3.27 (t, J=7.4 Hz, 2H), 2.49 (m, 10H), 2.28 (s, 3H), 2.03 (m, 2H).
Intermediate E7: General Procedure C

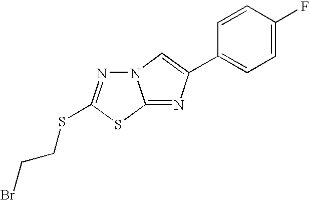

¹H NMR (200 MHz, DMSO-d⁶) δ8.67 (s, 1H), 7.85 (m, 2H), 7.23 (m, 2H), 3.78 (m, 4H).
Intermediate F7: General Procedure C

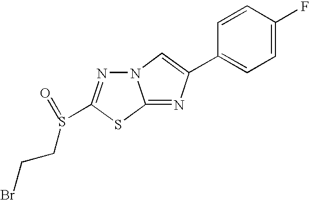

¹H NMR (200 MHz, CDCl₃) δ8.01 (s, 1H), 7.81 (m, 2H), 7.12 (m, 2H), 3.72 (m, 4H)
Compound 70:

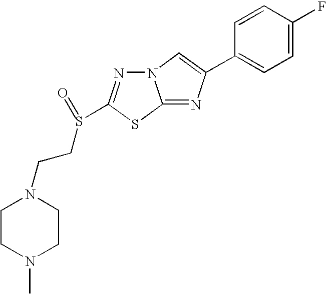

¹H NMR (200 MHz, CDCl₃) δ7.96 (s, 1H), 7.78 (m, 2H), 7.11 (t, J=8.6 Hz, 2H), 3.36 (t, J=6.0 Hz, 2H), 2.92 (m, 2H), 2.52 (s, 4H), 2.24 (s, 3H), 2.15 (2, 4H).
Intermediate E8: General Procedure C

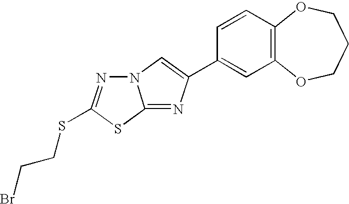

¹H NMR (200 MHz, CDCl₃) δ 7.84 (s, 1H), 7.34 (m, 2H), 6.97 (d, J=7.9 Hz, 1H), 4.21 (t, J=5.2 Hz, 4H), 3.64 (m, 4H), 2.18 (m, 2H).
Intermediate F8: General Procedure C

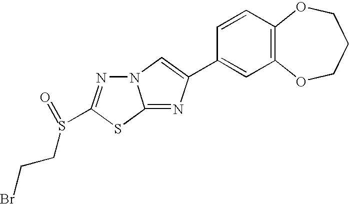

¹H NMR (200 MHz, CDCl₃) δ 7.95 (s, 1H), 7.42 (m, 2H), 7.01 (d, J=8.0 Hz, 1H), 4.24 (t, J=5.5 Hz, 4H), 3.71 (m, 4H), 2.21 (m, 2H).
Compound 71:

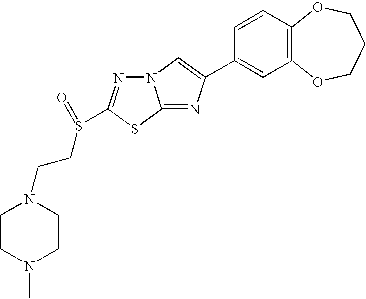

¹H NMR (200 MHz, CDCl₃) δ 7.92 (s, 1H), 7.43 (m, 2H), 7.02 (d, J=8.3 Hz, 1H), 4.25 (t, J=5.5 Hz, 4H), 3.36 (t, J=5.8 Hz, 2H), 2.92 (m, 2H), 2.50 (bs, 4H), 2.22 (m, 6H), 2.14 (s, 3H).

Compound 72:

Compound 72 was prepared using general method A.

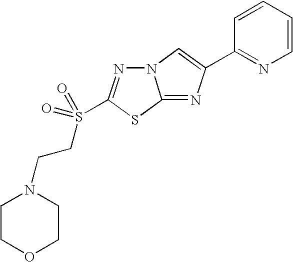

¹H NMR (200 MHz, DMSO-d⁶) δ 8.86 (s, 1H), 8.59 (d, J=4.9 Hz, 1H), 7.99 (m, 2H), 7.34 (m, 1H), 4.00 (t, J=5.8 Hz, 2H), 3.00 (br s, 4H), 2.72 (t, J=5.5 Hz, 2H), 2.23 (br s, 4H).

### EXAMPLE 74

### Protection of SCG Neurons from Anti-NGF Killing

SCG neurons were isolated from day 1 neonatal Sprague Dawley rats, plated at a cell density of 5,000 cells/well, and incubated in Biowhittaker Utraculture containing 1% Penstrep, 1% L-glutamine, 0.7% ARAC, 3% rat serum, and NGF (50 ng/mL, Calomone Labs), at 37° C., under a 5% CO₂ atmosphere. After 4 days the cells were treated with anti-NGF antibody (Sigma). At this time compound was added and the cells were maintained serum and NGF free for 48 hours, at which time viability was assessed using Alamar Blue (Medicorp) staining.

Table 1 summarizes selected IC₅₀ values from compounds tested using this protocol.

TABLE 1
Rescue from anti-NGF killing of SCG neurons.

| Compounds | IC(50) (μM) |
|---|---|
| 3 | 8 |
| 4 | 6 |

### EXAMPLE 75

### In Vitro Protection of SCG Neurons from Taxol Killing

SCG neurons were isolated from day 1 neonatal Sprague Dawley rats, plated at a cell density of 10,000 cells/well, and incubated in Biowhittaker Utraculture containing 1% Penstrep, 1% L-glutamine, 0.7% ARAC, 3% rat serum, and NGF (50 ng/mL, Calomone Labs) at 37° C., under a 5% CO₂ atmosphere. After 5 days the cells were treated with compound and Taxol™ (50 ng/mL). Viability was assessed 48 hours later using MTS (Promega) staining.

Table 2 summarizes selected IC₅₀ values from compounds tested using this protocol.

TABLE 2
Rescue from Taxol killing of SCG neurons.

| | IC₅₀ | | IC₅₀ | | IC₅₀ |
|---|---|---|---|---|---|
| Compounds | (μM) | Compound | (μM) | Compound | (μM) |
| 1 | 1 | 15 | 1 | 37 | 2 |
| 3 | 7 | 16 | 0.50 | 40 | 3 |
| 4 | 1 | 17 | 2 | 44 | 2 |
| 5 | 1 | 19 | 6 | 48 | 0.80 |
| 6 | 1.5 | 28 | 5 | 52 | 1 |
| 8 | 0.80 | 29 | 1 | 54 | 3 |
| 9 | 0.80 | 30 | >10 | 56 | 1 |
| 10 | 0.80 | 32 | 5 | 58 | 1 |
| 13 | 1 | 33 | 1 | 60 | 1 |
| 14 | 3 | 35 | 2 | 61 | 10 |
| 61 | 10 | 62 | 10 | 63 | 10 |
| 65 | 10 | 66 | 10 | 69 | 1 |
| 70 | 1 | 71 | 3 | | |

### EXAMPLE 76

### In Vitro Protection of SCG Neurons from Cisplatin Killing

SCG neurons were isolated from day 1 neonatal Sprague Dawley rats, plated at a cell density of 10,000 cells/well, and incubated in Biowhittaker Utraculture containing 1% Penstrep, 1% L-glutamine, 0.7% ARAC, 3% rat serum, and NGF (50 ng/mL, Calomone Labs) at 37° C., under a 5% CO₂ atmosphere. After 5 days the cells were treated with compound and cisplatin (3 μg/mL). Viability was assessed 48 hours later using MTS (Promega) staining.

TABLE 3
Protection of SCG neurons against cisplatin killing

| Entry | Compound | IC₅₀ (μM) |
|---|---|---|
| 1 | 2 | 0.8 |
| 2 | 16 | 0.5 |
| 3 | 17 | 0.3 |
| 4 | 18 | 0.3 |
| 5 | 19 | 0.8 |
| 6 | 28 | 1 |
| 7 | 29 | 3 |
| 8 | 44 | 2 |
| 9 | 45 | 1 |
| 10 | 69 | 1 |
| 11 | 70 | 1 |

### EXAMPLE

### In Vitro Killing of Human Neuroblastomas SK-N-AS and SH-SY5Y and Daoy Medulloblastoma Cells

Human neuroblastomas SK-N-AS, and SH-SY5Y were plated in 96 well plates at a density of 15 000 cells/well. Daoy medulloblastoma cells were plated at a density of 2000 cells/well. Cells were cultured in RPMI supplemented with 5% fetal bovine serum and test compound at various concentrations. After 72 hours of culture cell growth was assessed by sulforhodamine B. Cellular protein was precipitated and fixed with trichloroacetic acid and detected by incubation with sulforhodamine B. Bound dye solublized in trizma base and was assessed by absorbance at 515 nM.

1. A compound represented by Formula I: 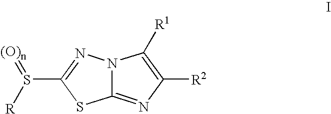
or a pharmaceutically acceptable salt thereof wherein:
n is 1 or 2;
R is selected from the group consisting of:
i. substituted or unsubstituted C (1-8) alkyl, fluoroalkyl, substituted and unsubstituted aralkyl, substituted and unsubstituted aryl, substituted and unsubstituted heteroaryl; and 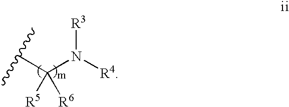
wherein m is an integer between 1 and 5; and
R³ and R⁴ are independently selected from the groups consisting of hydrogen, substituted or unsubstituted C (1-8) alkyl, fluoroalkyl, substituted and unsubstituted aralkyl, substituted and unsubstituted aryl, substituted and unsubstituted heteroaryl, substituted or unsubstituted C (1-8) alkylcarbonyl, substituted or unsubstituted C (1-8) alkylaminocarbonyl, substituted or unsubstituted arylaminocarbonyl, substituted or unsubstituted heteroarylaminocarbonyl, substituted or unsubstituted C (1-8) alkylsulfonyl, substituted or unsubstituted arylsulfonyl, substituted or unsubstituted heteroarylsulfonyl, or R³ and R⁴ are joined to form a substituted or unsubstituted cycloalkyl or cycloheteroalkyl ring system; and
R⁵ and R⁶ are independently chosen from hydrogen, substituted or unsubstituted C (1-8) alkyl, or R⁵ and R⁶ are joined to form a cycloalkyl or cycloheteroalkyl ring system;
R¹ and R² are independently selected from the group consisting of:
(i) H, fluoro C(1-6)-alkyl, substituted and unsubstituted C(6-16)-aryl, substituted and unsubstituted heteroaryl, substituted and unsubstituted coumarinyl, and adamantyl; 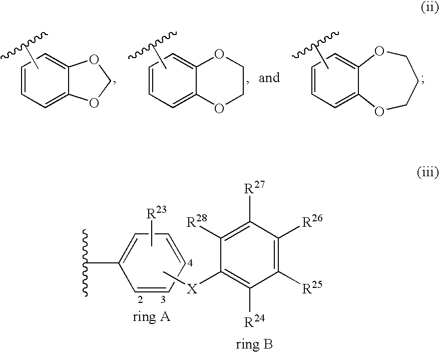
wherein
X is represented by a bond, O or S(O)ₙ, wherein n=0, 1, or 2, and is attached to ring A at the 2, 3, or 4 position;
R²³ on ring A is selected from the group consisting of H, halogen, C(1-8)alkyl, C(1-8) alkoxy and represents up to 4 substitutions;
R²⁴ through R²⁸ of ring B is independently selected from the group consisting of: H, halogen, C(1-8) alkyl, C(1-8) fluoroalkyl, C(1-8) alkoxy,
wherein any two adjacent R²⁴ through R²⁸ groups may be combined to form a fused aryl, substituted aryl, heteroaryl, or substituted heteroaryl ring system; and 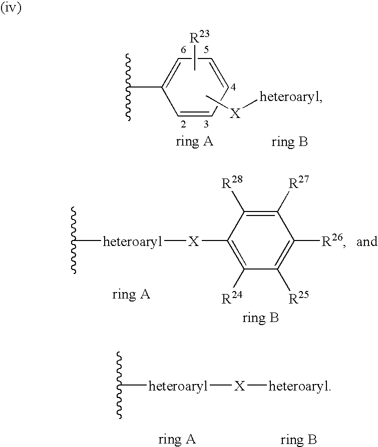
wherein
X is represented by a bond, O or S(O)ₙ, wherein n=0, 1, or 2;
R²³ on ring A is selected from the group consisting of: H, halogen, C(1-8) alkyl, C(1-8) alkoxy and represents up to 4 substitutions;
R²⁴ through R²⁸ of ring B are independently selected from the group consisting of: H, halogen, C(1-8) alkyl, C(1-8) fluoroalkyl, and C(1-8) alkoxy; and wherein any two adjacent R²⁴ through R²⁸ groups may be combined to form a fused aryl, substituted aryl, heteroaryl, or substituted heteroaryl ring system; and
wherein the heteroaryl ring systems of ring A and B contain at least one heteroatom and are substituted or unsubstituted.

2. The compound, according to claim 1, in which:
n=1; and
R is selected from the group consisting of:
i. substituted or unsubstituted C (1-8) alkyl, substituted and unsubstituted aralkyl, substituted and unsubstituted aryl, substituted and unsubstituted heteroaryl; and 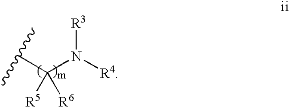
wherein m is an integer between 1 and 5; and
R³ and R⁴ are independently selected from the groups consisting of hydrogen, substituted or unsubstituted C (1-8) alkyl, substituted or unsubstituted C (1-8) alkylcarbonyl, or R³ and R⁴ are joined to form a substituted or unsubstituted cycloalkyl or cycloheteroalkyl ring system; and
R⁵ and R⁶ are independently chosen from hydrogen, substituted or unsubstituted C (1-8) alkyl.

3. The compound, according to claim 2, in which:
R is selected from the group consisting of:
i substituted or unsubstituted C (1-8) alkyl, substituted and unsubstituted aralkyl, substituted and unsubstituted aryl, substituted and unsubstituted heteroaryl; and 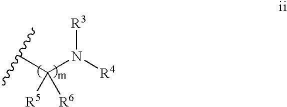
wherein m is 1, 2, or 3, and
R³ and R⁴ are independently selected from the groups consisting of hydrogen, substituted or unsubstituted C (1-8) alkyl, substituted or unsubstituted C (1-8) alkylcarbonyl, or R³ and R⁴ are joined to form a substituted or unsubstituted cycloheteroalkyl ring system; and
R⁵ and R⁶ are both hydrogen.

4. The compound, according to claim 1, in which:
n=1; and
R¹ and R² are independently selected from the group consisting of:
(i) H, substituted and unsubstituted C(6-16)-aryl, and substituted and unsubstituted heteroaryl; 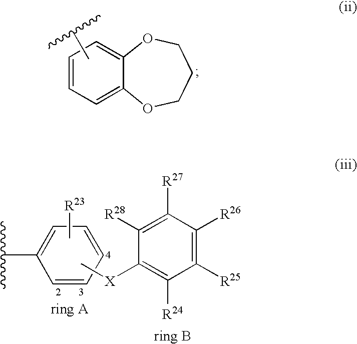
wherein
X is represented by a bond or 0 and is attached to ring A at the 2, 3, or 4 position;
R²³ on ring A is H and represents up to 4 substitutions;
R²⁴ through R²⁸ of ring B is independently selected from the group consisting of: H, halogen, C(1-8) fluoroalkyl, C(1-8) alkoxy.

5. The compound, according to claim 4, in which R¹ is H or phenyl.

6. The compound, according to claim 4, in which R² is independently selected from the group consisting of: H,

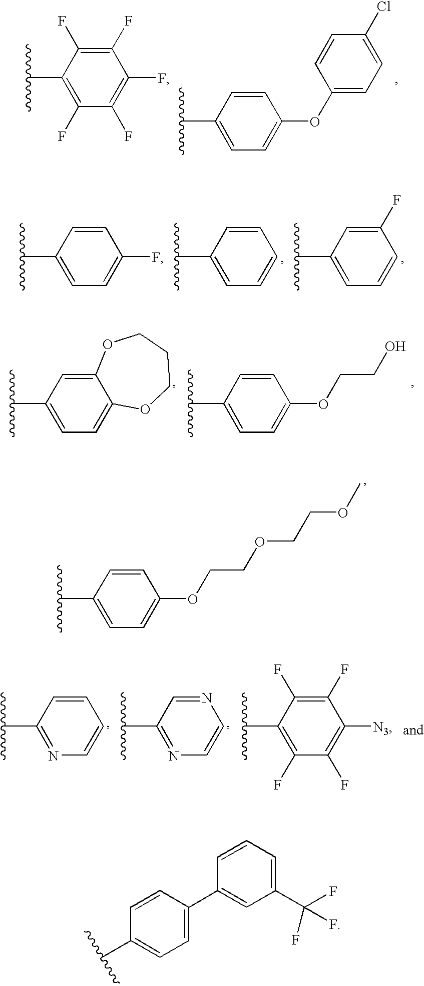

7. A compound of Formula I, where n=1 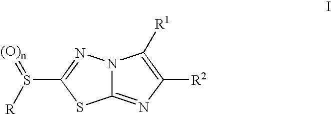
selected from the group consisting of:

| Compound | Structure |
|---|---|
| 1 | 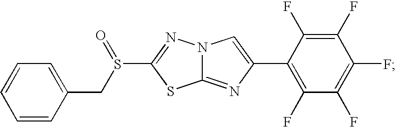 |
| 3 | 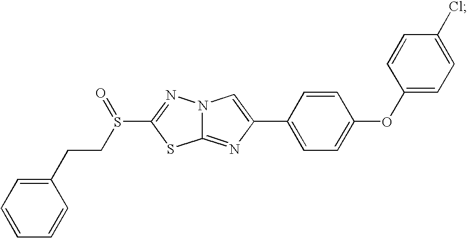 |
| 5 | 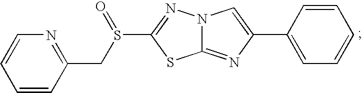 |
| 7 | 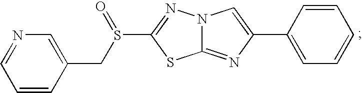 |
| 9 | 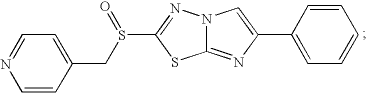 |
| 11 | 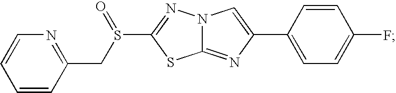 |
| 13 | 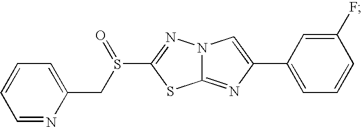 |
| 15 | 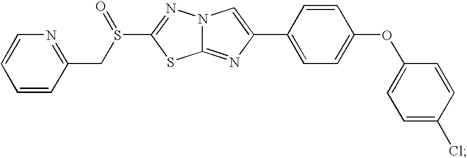 |
| 17 | 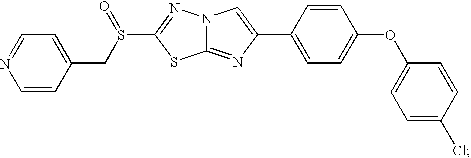 |
| 19 | 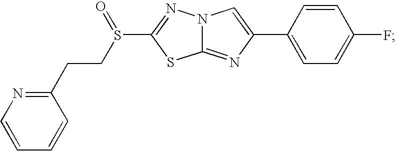 |
| 21 | 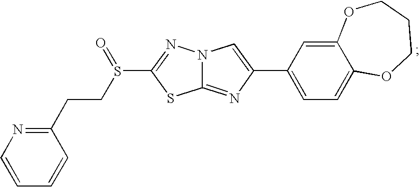 |
| 23 | 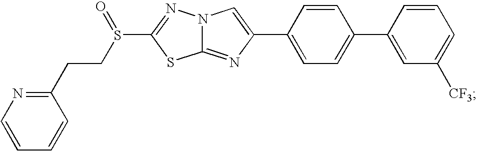 |
| 24 | 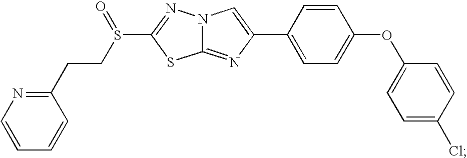 |
| 26 | 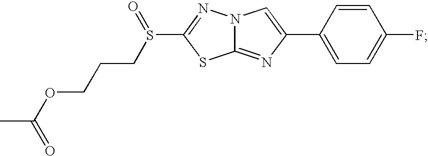 |
| 27 | 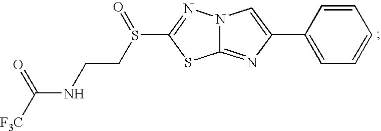 |
| 28 | 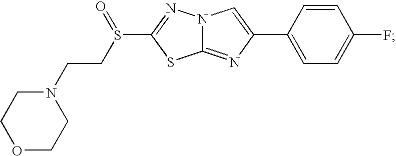 |
| 29 | 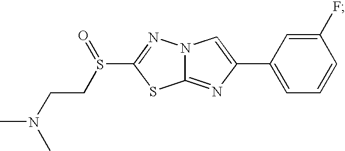 |
| 30 | 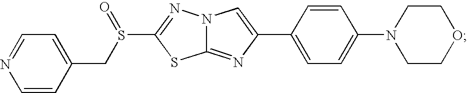 |
| 32 | 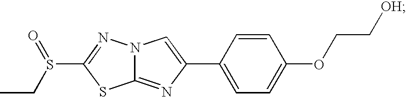 |
| 34 | 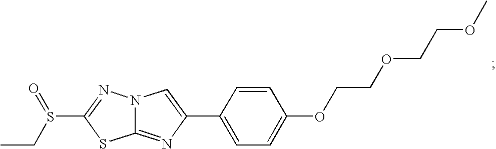 |
| 35 | 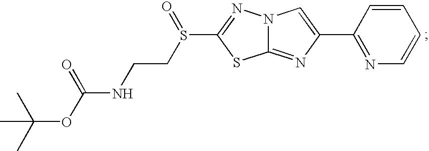 |
| 36 | 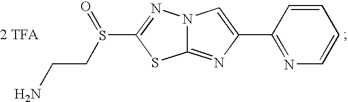 |
| 37 | 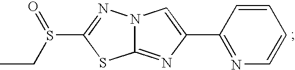 |
| 39 | 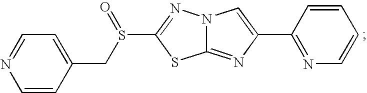 |
| 40 | 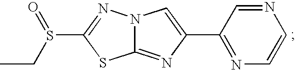 |
| 53 | 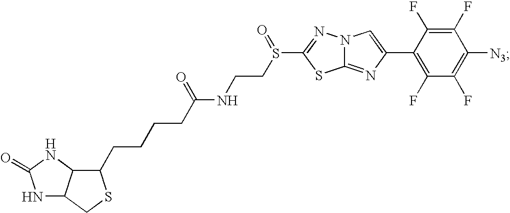 |
| 54 | 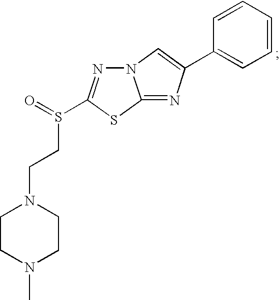 |
| 56 | 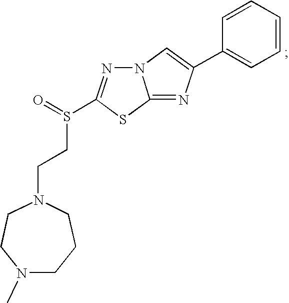 |
| 57 | 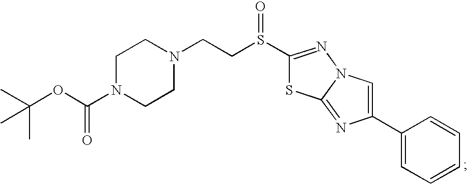 |
| 58 | 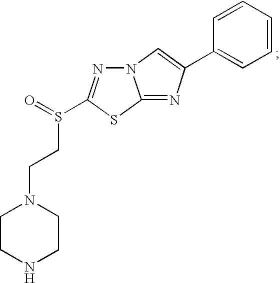 |
| 59 | 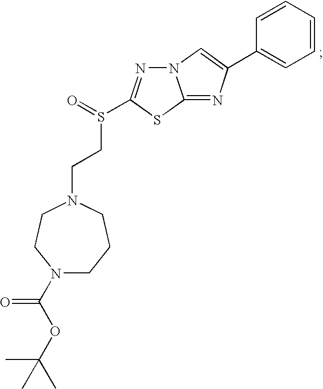 |
| 60 | 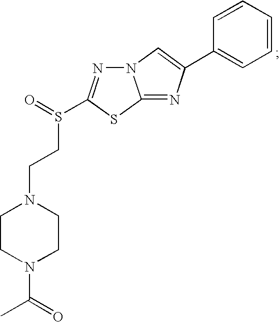 |
| 61 | 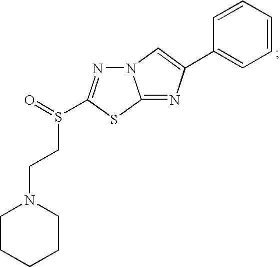 |
| 62 | 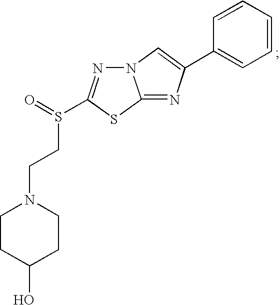 |
| 63 | 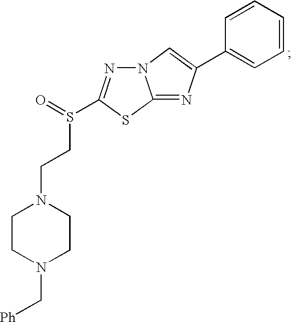 |
| 64 | 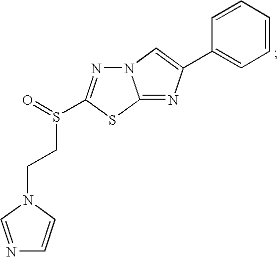 |
| 65 | 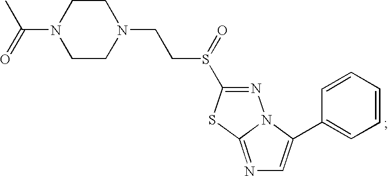 |
| 66 | 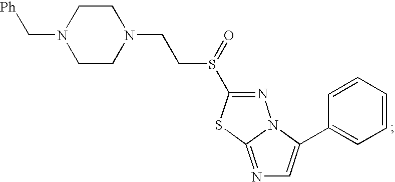 |
| 67 | 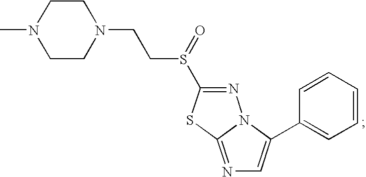 |
| 68 | 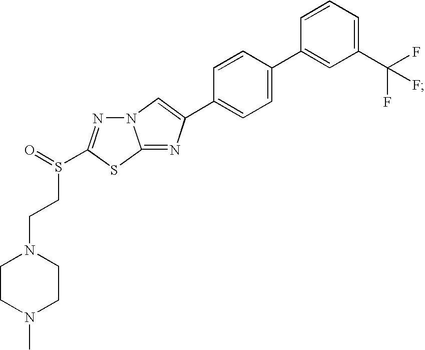 |
| 69 | 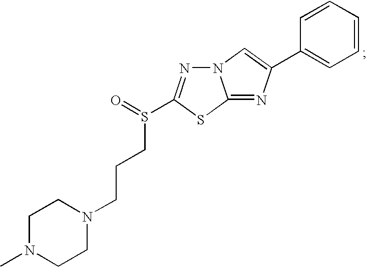 |
| 70 | 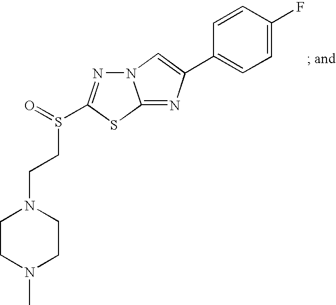 |
| 71 | 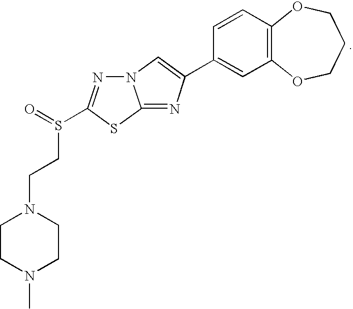 |

8. The compound, according to claim 7, in which compound 53 is a probe.

9. The compound, according to claim 1, in which:
n=2; and
R is selected from the group consisting of:
i. substituted or unsubstituted C (1-8) alkyl, substituted and unsubstituted aralkyl, and substituted and unsubstituted aryl; and 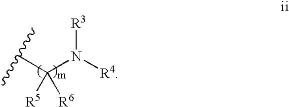
wherein m is an integer between 1 and 5; and
R³ and R⁴ are independently selected from the groups consisting of hydrogen, substituted or unsubstituted C (1-8) alkyl, substituted or unsubstituted C (1-8) alkylcarbonyl, substituted or unsubstituted C (1-8) alkylaminocarbonyl, substituted or unsubstituted C (1-8) alkylsulfonyl, or R³ and R⁴ are joined to form a substituted or unsubstituted cycloalkyl or cycloheteroalkyl ring system; and
R⁵ and R⁶ are independently chosen from hydrogen, substituted or unsubstituted C (1-8) alkyl.

10. The compound, according to claim 9, in which R is selected from the group consisting of:
i. substituted or unsubstituted C (1-8) alkyl, substituted and unsubstituted aralkyl, and substituted and unsubstituted aryl; and 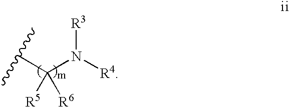
wherein m is an integer between 1, 2, and 3; and
R³ and R⁴ are independently selected from the groups consisting of hydrogen, substituted or unsubstituted C (1-8) alkyl, substituted or unsubstituted C (1-8) alkylcarbonyl, substituted or unsubstituted C (1-8) alkylaminocarbonyl, substituted or unsubstituted C (1-8) alkylsulfonyl, or R³ and R⁴ are joined to form a substituted or unsubstituted cycloheteroalkyl ring system; and
Both R⁵ and R⁶ are hydrogen.

11. The compound, according to claim 1, in which:
n=2; and
R¹ and R² are independently selected from the group consisting of:
(i) H, substituted and unsubstituted C(6-16)-aryl, and substituted and unsubstituted heteroaryl; and 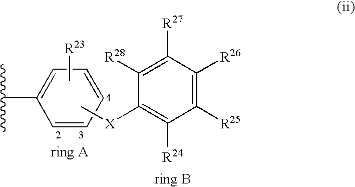
wherein
X is represented by a bond or O and is attached to ring A at the 2, 3, or 4 position;
R²³ on ring A is H and represents up to 4 substitutions; and
R²⁴ through R²⁸ of ring B is independently selected from the group consisting of: H, halogen, C(1-8) fluoroalkyl, and C(1-8) alkoxy.

12. The compound, according to claim 11, in which R¹ is H and R² is selected from the group consisting of:

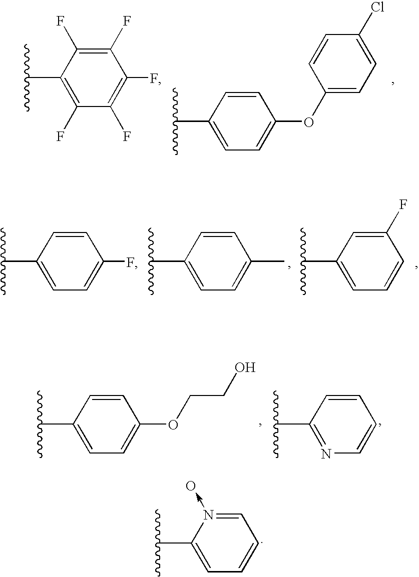

13. A compound of Formula I, where n=2 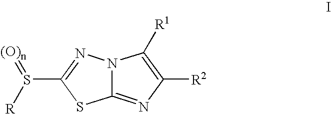
selected from the group consisting of:

| Compound | Structure |
|---|---|
| 2 | 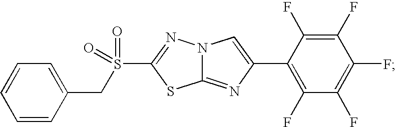 |
| 4 | 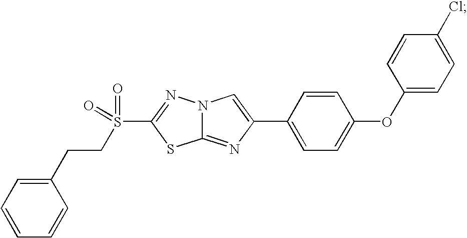 |
| 6 | 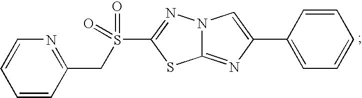 |
| 8 | 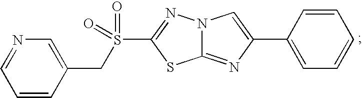 |
| 10 | 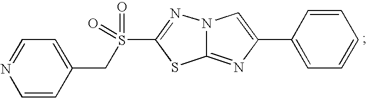 |
| 12 | 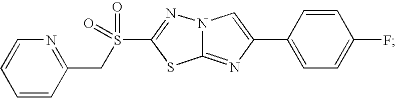 |
| 14 | 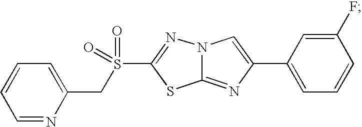 |
| 16 | 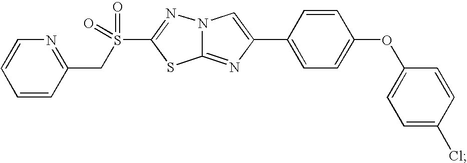 |
| 18 | 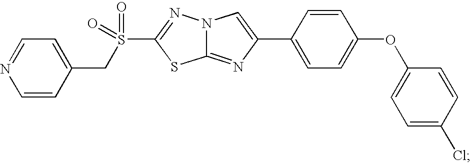 |
| 20 | 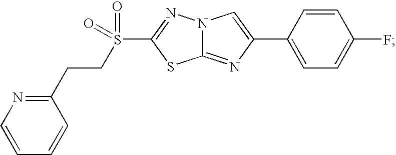 |
| 26 | 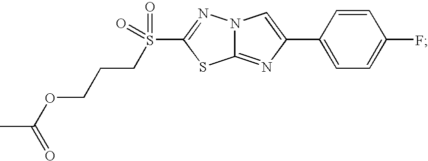 |
| 31 | 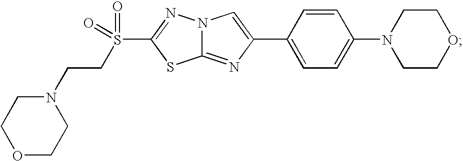 |
| 33 | 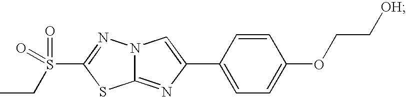 |
| 38 | 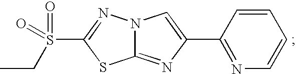 |
| 41 | 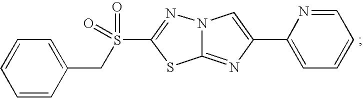 |
| 42 | 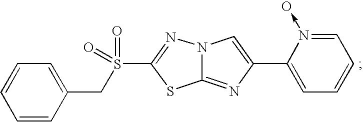 |
| 43 | 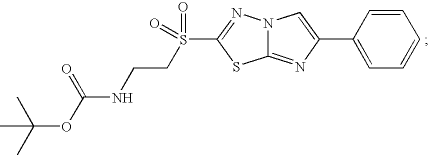 |
| 44 | 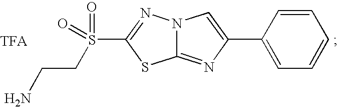 |
| 45 | 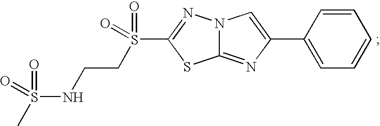 |
| 46 | 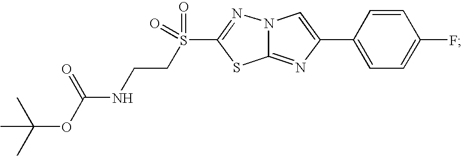 |
| 47 | 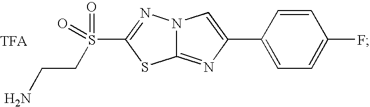 |
| 48 | 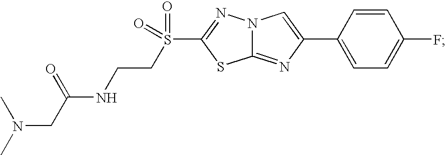 |
| 49 | 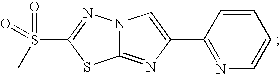 |
| 50 | 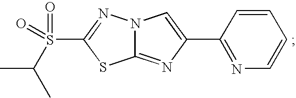 |
| 51 | 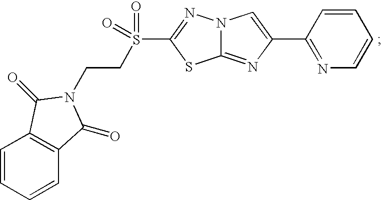 |
| 52 | 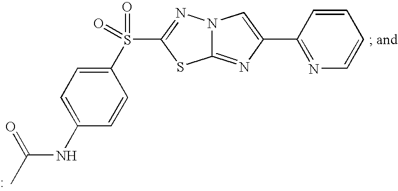 |
| 72 | 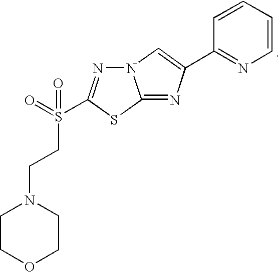 |

14. The compound, according to claim 1, in which the substituents are selected from the group consisting of:
1) H, halogen, nitro, cyano, C(1-8) alkyl, C(1-8) fluoroalkyl, aralkyl, aryl, heteroaryl, C(1-8) alkylcarbonyl, arylcarbonyl, heteroarylcarbonyl, azide, B(OH)₂, and adamantyl;
2) XR¹⁹ wherein X═O or S and R¹⁹ is C(1-8) alkyl, hydroxyl, C(1-4) alkoxy, fluoroalkyl, aryl, heteroaryl, lower alkylcarbonyl, arylcarbonyl, heteroarylcarbonyl, lower alkylaminocarbonyl, and arylaminocarbonyl; and
3) NR¹⁴R¹⁵ wherein R¹⁴ and R¹⁵ are each independently C(1-8) alkyl, or wherein R¹⁴ and R¹⁵ are joined to form an alkyl or heteroalkyl ring system,
wherein the C(1-8) alkyl, C(1-8) fluoroalkyl, aralkyl, aryl, heteroaryl, C(1-8) alkylcarbonyl, arylcarbonyl, heteroarylcarbonyl, and C(1-4) alkoxy may be further substituted by the substituents from 1), 2), and 3) above.

15. A composition comprising a compound, according to claim 1, together with a carrier.

16. A method of preventing or treating peripheral neuropathy in a subject, the method comprising administering to the subject in need thereof an effective amount of the composition, according to claim 15.

17. The method, according to claim 16, in which the peripheral neuropathy is induced by a toxic agent.

18. The method, according to claim 17, in which the toxic agent is a neurotoxic agent or a chemotherapeutic agent.

19. The method, according to claim 18, in which the chemotherapeutic agent is dideoxyinosine, deoxy cytizine, D4T, cisplatin, etoposide, vincristine, epithilone or its derivatives, Taxol™/Taxoter™ or derivatives thereof.

20. The method, according to claim 18, in which the neurotoxic agent is vincristine, vinblastine, cisplatin, Taxol™, D4T or other antivirals, dideoxy compounds, alcohol, metals, industrial toxins, overdoses of vitamins A, D or B6, penicillin or chloramphenicol.

21. A method of treating a neurodegenerative disease in a subject, the method comprising administering the subject in need thereof an effective amount of the composition, according to claim 15.

22. The method, according to claim 21, in which the neurodegenerative disease is Alzheimer's disease, Parkinson's disease, ALS, Huntington's disease, muscular dystrophy, diabetes, HIV, an ischemic insult, retinal ganglion loss following acute ocular stroke or glaucoma, a neurodegenerative condition resulting from a viral infection, and a neuropathy resulting from the use of chemotherapeutic agents used in the treatment of HIV.

23. The method, according to claim, 22 in which the neurodegenerative disease is a degenerative disease of the eye.

24. A method of treating a neurodegenerative disease in a subject, the method comprising co-administering to the subject in need thereof the composition, according to claim 15, with COX-2 inhibitors, NSAIDS, acetylcholinesterase inhibitors, L-dopa, ACE inhibitors or insulin.

25. A method of inducing axonal growth and/or repair in a subject, the method comprising administering the subject in need thereof an effective amount of the composition, according to claim 15.

26. A method of inducing axonal growth and/or repair in a subject, the method comprising administering to the subject in need thereof the composition, according to claim 15.

27. A method of altering signal transduction in a subject, the method comprising administering to the subject in need thereof the composition, according to claim 15.

28. A method of treating a proliferative disease in a subject, the method comprising administering to the subject in need thereof an effective amount of the composition, according to claim 15.

29. The method, according to claim 28, in which the proliferative condition is cancer.

30. The method, according to claim 29, in which the cancer is selected from the group consisting of prostate, colon, neuroblastoma, medulloblastoma, and breast cancer.

31. A method of treating a proliferative disease in a subject, the method comprising co-administering to the subject in need thereof the composition, according to claim 15, with a chemotherapeutic.

32. The method, according to claim 31, in which the chemotherapeutic is Taxol, cisplatin or vinca alkaloids.

34. An intermediate compound represented by Formula C1 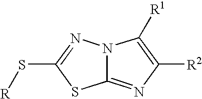
wherein R, R¹, and R² are as defined in claim 1.

35. A process for producing a compound of Formula I, according to claim 1, the process comprising:
oxidising an intermediate C1: 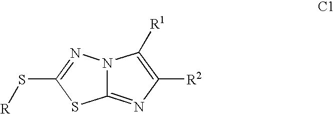
with an oxidising agent in a solvent so as to provide the compound of Formula I 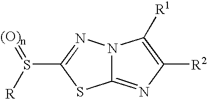
wherein n, R, R¹ and R² are as defined herein.

36. A process for producing a compound of Formula I, according to claim 1, the process comprising:
coupling intermediate B: 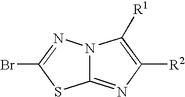
and a sodium sulfinate: 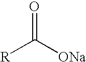
in a solvent so as to produce a compound of Formula I: 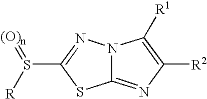
wherein n=2, and R, R¹ and R² are as defined herein.
